(19) United States
(12) Patent Application Publication
(10) Pub. No.: US 2023/0220075 A1
(43) Pub. Date: Jul. 13, 2023

(54) TSP-1 INHIBITORS FOR THE TREATMENT OF AGED, ATROPHIED OR DYSTROPHIED MUSCLE

(71) Applicant: The Board of Trustees of the Leland Stanford Junior University, Stanford, CA (US)

(72) Inventors: Helen M. Blau, Stanford, CA (US); Ermelinda Porpiglia, Stanford, CA (US); David M. Burns, Stanford, CA (US)

(73) Assignee: The Board of Trustees of the Leland Stanford Junior University, Stanford, CA (US)

(21) Appl. No.: 18/010,452

(22) PCT Filed: Jun. 22, 2021
(86) PCT No.: PCT/US2021/038549

§ 371 Date: Dec. 14, 2022

Related U.S. Application Data
(60) Provisional application No. 63/042,379, filed on Jun. 22, 2020.

(51) Int. Cl. C07K 16/28; A61P 21/00

(52) U.S. Cl. CPC C07K 16/2803; A61P 21/00; A61K 2039/505

(57) ABSTRACT

The present disclosure provides compositions and methods based on the inhibition of thrombospondin-1 as a therapeutic target in aging skeletal muscle to improve muscle mass, strength, function, maintenance, and regeneration.

### CROSS-REFERENCES TO RELATED APPLICATIONS

The present application claims priority to U.S. Provisional Pat. Appl. No. 63/042,379, filed on Jun. 22, 2020, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under grant numbers NS089533 and AG020961 awarded by the National Institutes of Health. The Government has certain rights in the invention.

### BACKGROUND

Skeletal muscles make up 40% of the body's mass. After the age of 50, humans lose on average 15% of their muscle mass per decade, culminating in the drastic loss of muscle strength characteristic of sarcopenia¹. This loss of strength with aging leads to diminished autonomy in the elderly and is associated with risk factors for disabling conditions, such as osteoporosis, heart failure, and cognitive decline²⁻⁴. There are currently no therapies for sarcopenia and the financial burden is high, approximating $19 billion in annual healthcare costs in the United States alone^{5,6}. Sarcopenia is known to be associated with a loss of functional skeletal muscle stem cells (MuSCs). MuSCs, also known as satellite cells, reside within skeletal muscle tissue in niches juxtaposed to myofibers and are required for skeletal muscle maintenance and regeneration throughout life⁷⁻¹⁴ Changes in cell extrinsic regulators, such as fibronectin, wnt, fibroblast growth factor-2 (FGF-2), and apelin in the muscle microenvironment diminish MuSC function with aging¹⁵⁻¹⁹. In addition, MuSCs isolated from aged mice exhibit intrinsic defects due to aberrant p38 MAPK, JAK/STAT and TGF-β signaling, which leads to a decline in the proportion of functional MuSCs, hindering muscle regeneration²⁰⁻²⁴. However, the absence of markers for distinguishing and prospectively isolating dysfunctional MuSCs has limited mechanistic insights and the development of therapies.

Accordingly, there is currently a need for novel methods and compositions for the identification of dysfunctional MuSCs and for enhancing the function of such cells, thereby increasing muscle regeneration and function in aged muscles. The present disclosure satisfies this need and provides other advantages as well.

### BRIEF SUMMARY

The present disclosure provides methods and compositions for enhancing MuSC function in aging, atrophied, or dystrophic skeletal muscle. The methods involve the inhibition of the interaction of thrombospondin-1 with CD47 on the surface MuSCs, and/or the activation of CD47 by thrombospondin. The methods are useful for the treatment of a large number of diseases and conditions associated with aging, atrophied, or dystrophic muscle.

Accordingly, in one aspect, the present disclosure provides a method of increasing muscle mass, strength, and/or regeneration in an aged, atrophied, or dystrophic skeletal muscle in a subject, the method comprising: administering to the aged, atrophied, or dystrophic skeletal muscle a thrombospondin-1 inhibitor in an amount sufficient to inhibit binding of thrombospondin-1 to CD47 on the surface of one or more muscle stem cells (MuSCs) and/or reduce thrombospondin-1 levels in one or more MuSCs in the aged, atrophied, or dystrophic skeletal muscle, thereby increasing muscle mass, strength, and/or regeneration in the aged, atrophied, or dystrophic skeletal muscle.

In some embodiments, the subject has sarcopenia. In some embodiments, the subject has one or more biomarkers of aging. In some embodiments, the one or more biomarkers of aging are selected from the group consisting of: decreased muscle mass and/or strength relative to a level present in young skeletal muscle, decreased MuSC proliferation or activation relative to a level present in young skeletal muscle, increased CD47 surface expression in MuSCs relative to a level present in young skeletal muscle, and decreased levels of Pax7 in MuSCs relative to a level present in young skeletal muscle.

In some embodiments, the subject has a condition or disease associated with muscle atrophy. In some such embodiments, the condition or disease is spinal muscular atrophy, diabetes, frailty, sarcopenic obesity, neuropathy, or cachexia, or wherein the subject has muscle atrophy due to immobilization or muscle disuse. In some embodiments, the subject has a muscular dystrophy. In some such embodiments, the muscular dystrophy is selected from the group consisting of Duchenne muscular dystrophy (DMD), Becker muscular dystrophy, congenital muscular dystrophy, distal muscular dystrophy, Emery-Dreifuss muscular dystrophy, facioscapulohumeral muscular dystrophy, limb girdle muscular dystrophy, myotonic muscular dystrophy (MDD), and oculopharyngeal muscular dystrophy.

In some embodiments, the aged, atrophied, or dystrophic skeletal muscle is injured. In some embodiments, the subject is preparing to undergo surgery, is undergoing surgery, or has undergone surgery. In some embodiments, the aged, atrophied, or dystrophic skeletal muscle is uninjured.

In some embodiments, the method results in an increase in muscle mass and/or regeneration relative to the level in the aged, atrophied, or dystrophic skeletal muscle prior to the administering of the thrombospondin-1 inhibitor. In some embodiments, the method results in an increase in muscle mass and/or regeneration of at least 10% relative to the level in the aged, atrophied, or dystrophic skeletal muscle prior to the administering of the thrombospondin-1 inhibitor. In some embodiments, the method results in an increase in muscle mass, strength, and/or regeneration in the aged, atrophied, or dystrophic skeletal muscle to a level substantially similar to a level present in young and/or non-dystrophic skeletal muscle.

In some embodiments, the administration results in an increase in the proliferation and/or activity of MuSCs in the aged skeletal muscle. In some embodiments, the administration results in an increase in the proliferation and/or activity of MuSCs in the aged, atrophied, or dystrophic skeletal muscle of at least 10% relative to the level in the aged, atrophied, or dystrophic skeletal muscle prior to the administering of the thrombospondin-1 inhibitor. In some embodiments, the administration results in an increase in the proliferation and/or activity of MuSCs in the aged, atrophied, or dystrophic skeletal muscle to a level substantially similar to a level present in young and/or non-dytrophic skeletal muscle.

In some embodiments, the administration results in a decrease in CD47 surface levels and/or an increase in Pax7 expression in MuSCs in the aged, atrophied, or dystrophic skeletal muscle. In some embodiments, the administration results in a decrease in CD47 surface levels and/or an increase in Pax7 expression in MuSCs in the aged, atrophied, or dystrophic skeletal muscle relative to the level in the aged, atrophied, or dystrophic skeletal muscle prior to the administering of the thrombospondin-1 inhibitor. In some embodiments, the administration results in a decrease in CD47 surface levels and/or an increase in Pax7 expression in MuSCs in the aged skeletal muscle of at least 10% relative to the level in the aged, atrophied, or dystrophic skeletal muscle prior to the administering of the thrombospondin-1 inhibitor. In some embodiments, the administration results in a decrease in CD47 surface levels in MuSCs in the aged, atrophied, or dystrophic skeletal muscle to a level substantially similar to a level present in young and/or non-dystrophic skeletal muscle.

In some embodiments, the subject is a human. In some embodiments, the human is over 30, 40, 50, 60, 70, or 80 years of age. In some embodiments, the human is selected for treatment with the inhibitor of thrombospondin-1 based on his or her age. In some embodiments, the subject is a non-human mammal. In some such embodiments, the non-human mammal is a farm animal.

In some embodiments, the inhibitor is a small molecule compound, a peptide, or a blocking antibody or antibody fragment. In some embodiments, the blocking antibody or antibody fragment is a monoclonal antibody or fragment thereof. In some embodiments, the antibody fragment is selected from the group consisting of Fab, F(ab′)₂, ScFv, diabody, and nanobody. In some embodiments, the inhibitor is an antisense oligonucleotide, microRNA, siRNA, shRNA, CRISPR gRNA, or messenger RNA.

In some embodiments, administering the inhibitor of thrombospondin-1 comprises systemic administration. In some embodiments, administering the inhibitor of thrombospondin-1 comprises local administration. In some embodiments, the local administration comprises intramuscular injection.

In another aspect, the present disclosure provides a method for regenerating a population of muscle cells in a subject having a condition or disease associated with muscle damage, injury, or atrophy, the method comprising: administering to the subject a therapeutically effective amount of an inhibitor of thrombospondin-1, to increase the population of muscle cells and/or to enhance muscle function in the subject.

In another aspect, the present disclosure provides a method for treating a condition or disease associated with muscle damage, injury or atrophy in a subject in need thereof, the method comprising: administering to the subject (i) a therapeutically effective amount of an inhibitor of thrombospondin-1, and a pharmaceutically acceptable carrier, and (ii) a population of isolated muscle cells, to treat the condition or disease associated with muscle damage, injury, or atrophy.

In another aspect, the present disclosure provides a method of treating muscle damage, muscle injury or muscle atrophy comprising: administering a therapeutically effective amount of a thrombospondin-1 inhibitor, to a subject in need by intramuscular administration.

In another aspect, the present disclosure provides a method of treating muscle damage, muscle injury or muscle atrophy comprising administering a therapeutically effective amount of a composition comprising a thrombospondin-1 inhibitor to a subject in need thereof, thereby treating said muscle damage, muscle injury or muscle atrophy.

In another aspect, the present disclosure provides a method for stimulating the proliferation of a population of isolated muscle cells, the method comprising: culturing the population of isolated muscle cells with a thrombospondin-1 inhibitor.

### BRIEF DESCRIPTION OF THE DRAWINGS

FIGS. 1A-1G. CD47 expression levels distinguish functionally and molecularly distinct aged muscle stem cell subsets. (FIG. 1A) Cell surface marker screening panel analysis of muscle stem cells (MuSCs). A single-cell suspension of Tibialis Anterior (TA) and Gastrocnemius (GA) muscle isolated from Pax7-ZsGreen reporter mice was stained with 176 cell surface antibodies and analyzed by fluorescence-based flow cytometry as described²⁶. MuSCs are identified as ZsGreen⁺ cells. The histogram overlay shows CD47 expression in ZsGreen⁺ cells. The filled gray histogram represents the isotype control. (FIG. 1B) CyTOF mass cytometry workflow. TA and GA muscles from young mice were triturated, digested to a single-cell suspension, stained with isotope-chelated antibodies and run through the CyTOF instrument. Stained cells were passed through an inductively coupled plasma, atomized, ionized, and the elemental composition was mass measured. Signals corresponding to each elemental tag were correlated to the presence of the respective isotopic marker. Gated Live/Lineage⁻/α₇integrin⁺/CD34⁺ MuSCs were analyzed with the X-shift algorithm (K=30 was auto-selected by the switch-point finding algorithm) yielding 2 clusters that were visualized using single-cell force-directed layout. Up to 2000 cells were randomly selected from each X-shift cluster, each cell was connected to 30 nearest neighbors in the phenotypic space and the graph layout was generated using the ForceAltas2 algorithm as previously described²⁶ (representative experiment, n=3 mice; 4 independent experiments). Expression levels of the myogenic transcription factor Pax7 (upper graph) and the surface marker CD47 (lower graph) were visualized (representative experiment, n=3 mice). (FIG. 1C) CD47 expression in young (2 months) and aged (24 months) MuSCs measured by single-cell RNA-seq analysis. Two-tailed unpaired t-test analysis with Welch's correction was used to determine the difference in CD47 expression between young and aged MuSCs. (FIG. 1D) CD47 protein expression in young and aged MuSCs measured by flow cytometry. TA and GA muscles were triturated, digested to a single-cell suspension, stained with fluorophore-conjugated antibodies to lineage markers (CD45, CD31, CD11b, Sca1), α₇integrin, CD34, CD47 and analyzed by fluorescence-based flow cytometry. MuSCs were identified as α₇integrin⁺/CD34⁺ cells. The histogram overlay shows CD47 expression in α₇integrin⁺/CD34⁺ MuSCs, from young (blue histograms) and aged (red histograms) mice (representative experiment, n=3 young mice, 3 aged mice). The filled gray histogram represents the isotype control. (FIG. 1E) Quantification of the relative abundance of MuSC subsets. Stacked columns indicate the relative proportion of each subset (CD47^{hi} in black. CD47^{lo} in white) within the Live/Lineage⁻/α₇integrin⁺/CD34⁺ MuSC population in muscle isolated from young and aged mice (mean±SEM from n=9 mice, 3 independent experiments). 2-way ANOVA analysis with Sidak correction for multiple comparisons was used to determine difference in the abundance of the individual subsets between young and aged MuSCs. (FIG. 1F) Scheme depicting the in vivo assay of regenerative capacity. Hindlimb muscles isolated from young and aged GFP⁺/Luciferase⁺ mice were digested to a single-cell suspension. CD47^{hi} and CD47^{lo} MuSC subsets were sorted and transplanted (50 cells/injection) into the TA muscle of hindlimb-irradiated NOD/SCID mice. Representative BLI images at 4 weeks post-transplant are shown (lower right panel). (FIG. 1G) Scatter plot shows the percentage of transplants from each condition that engrafted above threshold (dashed line, 80,000 photons/s) into recipient tissue and the BLI signal intensity (y axis). Line represents the median BLI signal (n=26 mice, 3 independent experiments). Kruskal Wallis test with significance determined by Dunn's multiple comparisons test was performed to determine the engraftment difference between CD47^{hi} and CD47^{lo} MuSC subset isolated from young or aged mice. *, ** and **** represent statistical significance at p≤0.05, p≤0.01 and p≤0.0001 respectively.

FIGS. 2A-2G. Alternative polyadenylation regulates CD47 expression at the onset of myogenic differentiation and is altered in aged muscle stem cells. (FIG. 2A) TA and GA muscles were isolated from young (2 months) and aged (24 months) mice, and digested to a single cell suspension that was stained using antibodies against lineage markers (CD45, CD11b, CD31, Sca1)-APCCy7, α7integrin-PE, CD9-APC and CD47-BV605. Cells were then fixed, permeabilized and stained for CD47 intracellularly using a different conjugate CD47-PECy7. A biaxial dot plot of CD47-PECy7 (intracellular) by CD47-BV605 (surface) is shown depicting the distribution of CD47 expression on the surface or inside the cell in stem cells from young (left panels) and aged (right panels) mice (representative sample, n=9 mice, 3 independent experiments). (FIG. 2B) The cells from each population in FIG. 2A are quantified as a fraction of total. Column bar graph indicates the relative proportion of each population (CD47−, CD47+ intracellular, CD47+ surface) within stem cells from young (left) and aged (right) mice. 2-way ANOVA analysis was used to determine the difference between young and aged populations. (FIG. 2C) The bar graph shows the expression level of CD47 protein on the surface of young and aged MuSCs (α7integrin+/CD34+), measured by flow cytometry as median fluorescence intensity (MFI) (mean±SEM from n=9 mice, 3 independent experiments). 1 tailed unpaired t-test analysis was used to determine the difference between young and aged stem cells. (FIG. 2D) Scheme depicting the CD47 coding sequence followed by the 3′ untranslated region (UTR). The 3′ UTR contains two functional polyadenylation signals (PAS): alternative polyA site selection generates messenger RNA (mRNA) transcripts of different length. RNA in situ hybridization probes were custom designed to differentiate between the short and long 3′UTR isoforms of CD47 mRNA using the PrimeFlow RNA assay. About 8000 fluorophores labeled each target mRNA. (FIG. 2E) TA and GA muscles were isolated from young (2 months) and aged (24 months) mice, and digested to a single cell suspension that was stained using antibodies against lineage markers (CD45, CD11b, CD31, Sca1)-APCCy7, α7 integrin-PE, CD9-APC and CD47-BV605. Cells were then fixed, permeabilized and stained for CD47 intracellularly using a different conjugate CD47-PECy7. Finally, cells were stained for the different CD47 mRNA isoforms using the custom-designed probes targeting the total or long 3′UTR isoform of CD47 mRNA and the PrimeFlow RNA assay kit according to manufacturer's instructions. The total and long 3′UTR isoform of CD47 mRNA were labeled with AF647 and AF750 respectively. A biaxial dot plot of CD47 mRNA-3′UTRₜₒₜₐₗ (y axis) by CD47 mRNA-3′UTR_{long} (x axis) is shown depicting the distribution of cells expressing the short isoform (upper left quadrant) or the long isoform (upper right quadrant) as a fraction of total CD47 mRNA (upper left and right quadrants combined) in stem cells from young (left panels) and aged (right panels) mice (representative sample, n=9 mice, 3 independent experiments). (FIG. 2F) Bar graph indicates the proportion of stem cells from young (left) and aged (right) mice expressing the CD47 mRNA with long 3′UTR. 2 tailed unpaired t-test analysis was used to determine difference in the fraction of stem cells expressing the CD47 mRNA with long 3′UTR between young and aged samples. (FIG. 2G) Bar graph shows the expression level of the CD47 mRNA with long 3′UTR in young and aged stem cells, measured by flow cytometry as median fluorescence intensity (MFI). 2 tailed paired t-test analysis was used to determine difference in the expression level of CD47 mRNA with long 3′UTR between young and aged stem cells. * and *** represent statistical significance at p≤0.05 and p≤0.001 respectively.

FIGS. 3A-3L. Aberrant thrombospondin-1 signaling via CD47 inhibits the proliferative capacity of aged muscle stem cells. (FIG. 3A) Thrombospondin-1 (THBS1) expression in young and aged MuSCs measured by bulk RNA seq analysis (n=3 young mice, n=5 aged mice). Two-tailed unpaired t-test analysis with Welch's correction was used to determine the difference in THBS1 expression between young and aged MuSCs. (FIG. 3B) Sorted α₇integrin⁺/CD34⁺ MuSCs from young wild type of CD47^{-/-} mice were cultured for one week in the presence of increasing doses of THBS1 (0.6 μg/ml-5 μg/ml) and proliferation was quantified by cell count. (FIG. 3C) Fraction of IdU⁺ MuSCs measured by CyTOF analysis in response to a 6-day treatment of young MuSCs with increasing doses of thrombospondin-1. Line represents the mean±SD (n=5 young mice). 2-way ANOVA analysis with Tukey correction for multiple comparisons was used to determine difference in the fraction of IdU⁺ cells between untreated and thrombospondin-treated young MuSCs. (FIG. 3D) Fraction of IdU⁺ MuSCs measured by CyTOF analysis in response to a 6-day treatment of sorted wild type (young, aged) and CD47^{-/-} aged MuSCs with a blocking antibody to thrombospondin-1. Line represents the mean±SD (n=3 wild type young mice, 3 wild type aged mice, 3 aged CD47^{-/-} mice). Two-tailed paired t test was performed to determine the difference in the fraction of IdU⁺ cells between anti-THBS1 treated and control sorted MuSCs from young, aged or CD47^{-/-} mice. (FIGS. 3E, F) Quantification of MuSC subset expansion after THBS1 blockade. The CD47^{lo} and CD47^{hi} MuSCs subsets were sorted from young (FIG. 3E) and aged (FIG. 3F) mice and cultured in growth media for 6 days on biomimetic hydrogels in the presence (+) or absence (−) of a blocking antibody to THBS1. Cell number was quantified by cell count at day 0 (D0) or at day 6 (D6) after culture (representative images, n=7 young replicates, n=3 aged replicates). 2-way ANOVA analysis with Tukey correction for multiple comparisons was used to determine difference between conditions in CD47^{lo} and CD47^{hi} subsets isolated from young (FIG. 3E) or aged (FIG. 3F) mice. (FIG. 3G) (Top scheme) THBS1-CD47-cAMP signaling axis. THBS1-CD47 signals through Guanine nucleotide-binding protein Gᵢ's alpha subunit which inhibits adenylyl cyclases to reduce cAMP levels. (Bottom scheme) The cADDis downward sensor is a fluorescent cAMP sensor capable of detecting changes in cAMP concentration in living cells. cAMP binding to the cADDis downward sensor reduces GFP fluorescence. (FIG. 3H) CD47^{lo} and CD47^{hi} MuSCs were sorted from young mice and transfected overnight with a baculovirus encoding the cAMP sensor. The following day MuSC subsets were treated with THBS1 and individually imaged with a confocal microscope at 10 sec intervals for 5 minutes. The graph shows the average normalized GFP fluorescence of CD47^{lo} (gray) and CD47^{hi} (white) MuSCs over time. Two-tailed unpaired t-test analysis was used to determine the difference in normalized fluorescence expression between CD47^{lo} and CD47^{hi} MuSC subsets after stimulation with THBS1. (FIG. 3I) CyTOF analysis of young hindlimb muscle showing α₇integrin⁺/CD34⁺ MuSCs from young mice. Representative biaxial dot plots of THBS1 by CD47 colored by population density shows a CD47^{hi} MuSC subset that expresses high levels of THBS1. (FIG. 3J) CyTOF analysis of young hindlimb muscle showing the entire myogenic compartment defined as Lin⁻/α₇ integrin⁺/CD9⁺ cells. Representative biaxial dot plots of CD9 (y axis) by CD104 (x axis) colored by THBS1 expression (left) and CD47 expression (right) highlights that the progenitor population P1 expresses the highest level of both THBS1 and CD47. (FIGS. 3K, 3L) Intracellular THBS1 protein measurement by CyTOF during regeneration. Mice were acutely injured by notexin injection in the TA and GA muscles, 6 or 3 days prior to tissue collection. Muscle tissues of the indicated groups were simultaneously collected at day 0, stained with isotope-chelated antibodies, run through the CyTOF instrument and analyzed. Violin plots show THBS1 protein levels in SC (left) and P1 (right) population from young (FIG. 3K) and aged (FIG. 3L) mice during an injury time course. Kruskal Wallis test with significance determined by Dunn's multiple comparisons test was performed to determine the difference in THBS1 expression between the indicated timepoints in SC or P1 cells from young (FIG. 3K) or aged (FIG. 3L) mice. *, ** and **** represent statistical significance at p≤0.05, p≤0.01 and p≤0.0001 respectively.

FIGS. 4A-4J. Thrombospondin-1 blockade in vivo activates muscle stem cells in absence of injury. (FIG. 4A) Experimental scheme (upper panel). Endogenous MuSC expansion was assayed by bioluminescence imaging (BLI) in Pax7^{CreERT2}; Rosa26-LSL-Luc mice treated with tamoxifen (TAM) (n=7 mice per condition). Mice were injected intramuscularly in the TA and GA muscles (three times, at two-days interval) with an antibody to THBS1 or a control IgG (contralateral leg) and imaged by BLI. The graph shows the summary of the BLI signal intensity (y axis) over time for each group (light blue, treatment with thrombospondin-1 blocking antibody; gray, treatment with IgG control). Multiple t-tests with Holm-Sidak correction for multiple comparisons were used to determine the difference in bioluminescence signal between IgG control and anti-THBS1 treated samples at the indicated time points. (FIG. 4B) Experimental scheme (upper panel). Young and aged mice were treated by intramuscular injections in the TA and GA muscles with a blocking antibody to THBS1 as in (FIG. 4A) and hindlimb muscle tissue was collected for CyTOF analysis at the end of treatment. (Left) Representative biaxial dot plots of CD98 (y axis) by CD44 (x axis) colored by channel show IdU incorporation in activated MuSCs, defined by co-expression of surface markers CD98 and CD44 (upper right quadrant). (Upper right) Bar graph indicates the proportion of CD98⁺/CD44⁺ activated MuSCs. 2 tailed paired t-test was used to determine the difference in the abundance of CD98⁺/CD44⁺ subset between IgG control and anti-THBS1 treatment in young or aged samples. (Lower right) Bar graph shows IdU⁺ cells (%) in the MuSC population (lower right). 2 tailed paired t-test was used to determine the difference in the abundance of IdU⁺ stem cells between IgG control and anti-THBS1 treated samples in young or aged samples. (FIG. 4C) Experimental scheme. Young and aged mice were treated with a blocking antibody to THBS1 as in (FIG. 4B) and hindlimb muscle tissue was collected for CyTOF analysis, 6 days after the last THBS1 blocking antibody or IgG injection. (FIG. 4D) Live/Lineage⁻/α₇integrin⁺/CD9⁺ cells gated were clustered using the X-shift algorithm (K=50 was auto-selected by the switch-point finding algorithm) yielding 61 clusters. Up to 10,000 cells were randomly selected from each X-shift cluster, each cell was connected to 50 nearest neighbors in the phenotypic space and the graph layout was generated using the ForceAtlas2 algorithm (n=3 mice for each condition). Identified clusters were visualized using single-cell force-directed layout. The expression levels of the myogenic transcription factor Pax7 (upper panels) and the surface marker CD47 (lower panel) were visualized and are shown in the panel composite. (FIG. 4E) The floating bar graph shows the change in the proportion of each MuSC subset in aged mice treated with THBS1 blockade, relative to IgG control. (FIG. 4F) Bar graph shows the fraction of Pax7⁺ cells in the Live/Lineage⁻/α₇integrin⁺/CD9⁺ cell population from young control mice and aged control and treated mice. Unpaired t-test was used to determine the difference in the proportion of Pax7⁺ cells within Live/Lineage⁻/α₇integrin⁺/CD9⁺ cell population between young and aged IgG control. Paired t-test was used to determine the difference in the proportion of Pax7⁺ cells within Live/Lineage⁻/α₇integrin⁺/CD9⁺ cell population between IgG control and anti-THBS1 treatment in aged samples. (FIG. 4G) Young mice were pretreated (three times, at two-days intervals) by intramuscular injection in the TA and GA muscles with a blocking antibody to THBS1 or IgG control (contralateral leg) and hindlimb muscle tissue was collected for histology 10 days after the end of treatment. Myofiber cross sectional area (CSA) was quantified in IgG treated (gray) and anti-THBS1 treated (light blue) TA muscles and curve fitting was performed (n=7 per group). Chi-square test was performed to determine the difference in the CSA distribution between IgG control and anti-THBS1 treated young TA muscles. (FIG. 4H) The scatter plot shows the mean CSA in sectioned IgG treated (gray) and anti-THBS1 treated (light blue) TA muscles 10 days after the end of treatment. Paired t-test was used to determine the difference in CSA between IgG control and anti-THBS1 treated young TA muscles. (FIG. 4I) The scatter plot shows the fraction of Pax7⁺ cells in IgG treated (gray) and anti-THBS1 treated (light blue) TA muscles 10 days after the end of treatment. Paired t-test was used to determine the difference in the number of Pax7⁺ cells between IgG control and anti-THBS1 treated young TA muscles. (FIG. 4J) Wild type young and aged mice, and young CD47^{-/-} mice were pretreated (three times, at two-days intervals) by intramuscular injection in the TA and GA muscles with a blocking antibody to THBS1 or IgG control (contralateral leg) and grip strength was measured 10 days after the end of treatment. Paired t-test was used to determine the difference in grip strength between IgG control and anti-THBS1 treatment in young wild type, aged wild type and young CD47^{-/-} samples 10 days after the end of treatment. Paired t-test was used to determine the difference in grip strength and tetanic force between IgG control and anti-THBS1 treatment in young wild type, aged wild type and young CD47^{-/-} samples 10 days after the end of treatment. *, ** and **** represent statistical significance at p≤0.05, p≤0.01 and p≤0.0001 respectively.

FIGS. 5A-5E. Thrombospondin-1 blockade in vivo enhances the regenerative response of aged muscle leading to increased strength. (FIG. 5A) Experimental scheme. Young and aged mice were pretreated (three times, at two-days intervals) by intramuscular injection in the TA and GA muscles with a blocking antibody to THBS1 or IgG control (contralateral leg) prior to notexin injury and hindlimb muscle tissue was collected for CyTOF analysis 3- or 6-days post injury. (FIG. 5B) The scatter plot shows the proportion of MuSCs within the myogenic compartment for each condition, at day 3 post injury. Paired t-test was used to determine the difference in the abundance of MuSCs between IgG control and anti-THBS1 treatment in young or aged samples at day 3 post injury. (FIG. 5C) (Left) Representative biaxial dot plots of CD98 by CD44 colored by channel, show IdU incorporation in activated MuSCs, defined by co-expression of surface markers CD98 and CD44 (upper right quadrant). (Upper right) The scatter plot shows the proportion of CD98+/CD44+ activated MuSCs. 2 tailed paired t-test was used to determine the difference in the abundance of CD98+/CD44+ subset between IgG control and anti-THBS1 treatment in young or aged samples. (Lower right) The scatter plot shows IdU+ cells (%) in the MuSC population. (FIG. 5D-5E) Wild type young and aged mice, and young CD47−/− mice were pretreated (three times, at two-days intervals) by intramuscular injection in the TA and GA muscles with a blocking antibody to THBS1 or IgG control (contralateral leg) prior to notexin injury and grip strength (FIG. 5D) and tetanic force (FIG. 5E) were measured at 10 days post injury. Paired t-test was used to determine the difference in grip strength and tetanic force between IgG control and anti-THBS1 treatment in young wild type, aged wild type and young CD47−/− samples at day 10 post injury. *, ** and **** represent statistical significance at p≤0.05, p≤0.01 and p≤0.0001, respectively.

FIGS. 6A-6D. (FIG. 6A) CyTOF analysis was performed on TA and GA hindlimb muscle. Gated Live/Lineage⁻/α₇integrin⁺/CD34⁺ MuSCs were analyzed with the X-shift algorithm (K=30 was auto-selected by the switch-point finding algorithm) yielding 3 clusters (colour-coded in red, green and blue) that were visualized using single-cell force-directed layout. Up to 2000 cells were randomly selected from each X-shift cluster, each cell was connected to 30 nearest neighbors in the phenotypic space and the graph layout was generated using the ForceAltas2 algorithm as previously described²⁶ (representative experiment, n=3 mice; 4 independent experiments). (FIG. 6B) Quantification of the expression of myogenic transcription factors Pax7, Myf5, MyoD and Myogenin in CD47^{lo} and CD47^{hi} MuSC subsets, measured by CyTOF analysis as median intensity (mean±SEM from n=9 mice, 3 independent experiments). (FIG. 6C) Quantification of CD47 protein expression, measured by flow cytometry as median fluorescence intensity (MFI), in young (2 months) and aged (24 months) MuSCs (Lin⁻/α₇integrin⁺/CD34⁺), CD47^{lo} and CD47^{hi} MuSC subsets (mean±SEM from n=9 mice, 3 independent experiments). 2-way ANOVA analysis with Tukey correction for multiple comparisons was used to determine difference between young and aged subsets. *** represent statistical significance at p≤0.001. (FIG. 6D) Stem cell repopulation analysis in primary recipients performed by flow cytometric detection of donor-derived (GFP⁺) MuSCs as a fraction of the total recipient MuSC (integrin α₇+/CD34⁺) population, 4 weeks after transplantation. *** represent statistical significance at p≤0.001.

FIGS. 7A-7K (FIG. 7A) TA and GA muscles were isolated from young and aged mice and digested to a single cell suspension that was stained using antibodies against lineage markers (CD45, CD11b, CD31, Sca1)-APCCy7, α7 integrin-PE, CD9-APC and CD47-BV605. Cells were then fixed, permeabilized and stained for CD47 intracellularly using a different conjugate CD47-PECy7. A biaxial dot plot of CD47-PECy7 (intracellular) by CD47-BV605 (surface) is shown depicting the distribution of CD47 expression on the surface or inside the cell in progenitor cells from young (left panels) and aged (right panels) mice (representative sample, n=9 mice, 3 independent experiments). (FIG. 7B) The cells from each population in (FIG. 7A) are quantified as a fraction of total. Column bar graph indicates the relative proportion of each population (CD47⁻, CD47⁺ intracellular, CD47⁺ surface) within progenitor cells from young (left) and aged (right) mice. 2-way ANOVA analysis was used to determine difference between young and aged subsets. (FIG. 7C) The mRNA sequence of CD47 from mouse, human, gorilla and dog were aligned using MAFFT online. 3 highly conserved polyadenylation sites (PAS) were identified (red boxes). (FIG. 7D) Quantification of murine CD47 mRNA isoform abundance by analysis of publicly available datasets obtained by 3′ region extraction and deep sequencing³⁸. Bar graph shows the abundance of the different CD47 mRNA isoforms. Polyadenylation site 1 (PAS)=chr16:49911190-49911205; PAS2=chr16:49,912530-49,912555; PAS3=chr16: 49,915032-49,915123. (FIG. 7E) Gating strategy used to identify stem and progenitor cells on flow cytometry samples described in FIG. 7E and FIGS. 7G-7J. Individual dot plots are shown. (FIG. 7F) Expression of CD47 mRNA measured by qPCR in muscle cells from wild type and CD47^{-/-} quadriceps muscles. 2 tailed unpaired t-test analysis was used to determine difference between wild type and CD47^{-/-} samples. (FIG. 7G) TA and GA muscles were isolated from wild type and CD47^{-/-} mice, and digested to a single cell suspension that was stained using antibodies against lineage markers (CD45, CD11b, CD31, Sca1)-APCCy7, α7 integrin-PE. CD9-APC and CD47-BV605. Cells were then fixed, permeabilized and stained for CD47 intracellularly using a different conjugate CD47-PECy7. Finally, cells were stained according to the PrimeFlow RNA protocol for the different CD47 mRNA isoforms A biaxial plot of CD47 mRNA-3′UTRₛₕₒᵣₜ (y axis) by CD47 mRNA-3′UTR_{long} (x axis) is shown depicting the distribution of cells expressing the CD47 mRNA short isoform only (upper left quadrant) or simultaneously the short and long isoforms (upper right quadrant) in progenitor cells from wild type (blue contour plot) and CD47^{-/-} (gray dots) mice. (FIG. 7H) Bar graph shows the fold change in the expression level of the CD47 mRNA with short (left) or long (right) 3′UTR, measured by flow cytometry as median fluorescence intensity (MFI), in CD47^{-/-} progenitor cells compared to wild type. (FIG. 7I) TA and GA muscles were isolated from young (2 months) and aged (24 months) mice, digested and stained as in (FIG. 7G). A biaxial dot plot of CD47 mRNA-3′UTRₜₒₜₐₗ (y axis) by CD47 mRNA-3′UTR_{long} (x axis) is shown depicting the distribution of cells expressing the CD47 mRNA short isoform (upper left quadrant) or the long isoform (upper right quadrant) as a fraction of total CD47 mRNA (upper left and right quadrants combined) in progenitor cells from young (left panels) and aged (right panels) mice (representative sample, n=9 mice, 3 independent experiments). (FIG. 7J) Bar graph indicates the proportion of progenitor cells from young (left) and aged (right) mice expressing the long 3′UTR. 2 tailed unpaired t-test analysis was used to determine difference between young and aged stem cells. **** represents statistical significance at p≤0.0001. (FIG. 7K) Abundance of CD47 mRNA with long 3′ UTR measured by RT-PCR in young and aged sorted muscle stem and progenitor cells, and normalized to the expression in young stem cells. Bar graph represents the mean±SEM (n=3). 2-way ANOVA analysis with Tukey correction for multiple comparisons was used to determine difference in the abundance of CD47 mRNA with long 3 UTR across the different conditions in young or aged sorted muscle stem and progenitor cells.

FIGS. 8A-8G. (FIGS. 8A-8C) Fraction of ki-67⁺ (FIG. 8A), Cleaved PARP⁺ (FIG. 8B), p57⁺ (FIG. 5C) MuSCs, measured by CyTOF analysis in response to a 6-day treatment of young MuSCs with increasing doses of thrombospondin-1. Line represents the mean±SD (n=5 young mice). 2-way ANOVA analysis with Tukey correction for multiple comparisons was used to determine difference in the fraction of ki-67⁺ (FIG. 8A), Cleaved PARP⁺ (FIG. 8B), p57⁺ (FIG. 5C) cells between untreated and thrombospondin-1 treated young MuSCs. (FIG. 8D) Expression levels of Cdkn1a, Cdkn1b, Cdkn1c, measured by q-RT-PCR in sorted MuSCs treated with thrombospondin-1 for 21 h. Line represents the mean±SEM (3 independent experiments). Two-tailed unpaired t test was performed to determine the difference in gene expression levels between thrombospondin-1 treated MuSCs and control treated MuSCs. (FIG. 8E) α₇integrin/CD34⁺ MuSCs sorted from young and aged mice were cultured in growth media for 6 days on biomimetic hydrogels in the presence (+) or absence (−) of a blocking antibody to THBS1 and proliferation was quantified by cell count (meant SEM from n=7 young replicates, n=3 aged replicates). 2-way ANOVA analysis with Tukey correction for multiple comparisons was used to determine difference between conditions in young and aged MuSCs. (FIG. 8F) MuSCs sorted from young wild type and CD47^{-/-} mice were cultured in growth media for 6 days on biomimetic hydrogels in the presence (+) or absence (−) of a blocking antibody to THBS1 and proliferation was quantified by cell count (mean±SEM from n=4 young replicates, n=4 aged replicates). 2-way ANOVA analysis with Tukey correction for multiple comparisons was used to determine difference between conditions in wild type and CD47^{-/-} MuSCs. (FIG. 8G) CyTOF analysis of young hindlimb muscle showing α₇integrin⁺/CD34⁺ MuSCs during an injury time course. Representative biaxial dot plots of CD47 (y axis) by α7 integrin (x axis) colored by channel shows Pax7 expression (upper panels) and IdU incorporation (lower panels) within CD47^{hi} and CD47^{lo} MuSC subsets during the injury time course (Day 0, Day 3, Day 6). (FIG. 8H) Quantification of the proportion of CD47^{lo} and CD47^{hi} MuSCs during the injury time course in (FIG. 8G). *, ** and **** represent statistical significance at p≤0.05, p≤0.01 and p≤0.0001 respectively.

FIGS. 9A-9B. (FIG. 9A) Young and aged mice were treated (three times, at two-day intervals) with a blocking antibody to THBS1 or IgG control (contralateral leg) prior to notexin injury and hindlimb muscle tissue was collected for CyTOF analysis 6 days post injury. Scatter plot shows the fraction of Pax7⁺ cells in the Live/Lineage⁻/α₇integrin⁺/CD9⁺ cell population from young control mice and aged control and treated mice at day 6 post injury. Paired t-test was used to determine the difference in the abundance of Pax7⁺ MuSCs between IgG control and anti-THBS1 treatment in aged samples. (FIG. 9B) Model. Skeletal muscle injury leads to MuSC activation. We found that in young muscle during regeneration, progenitor cells participate in a negative feedback loop whereby they produce THBS1 to limit MuSC proliferation, and promote MuSC return to quiescence, therefore preventing MuSC exhaustion. We discovered that in aged muscle accumulation of a dysfunctional CD47^{hi} MuSC subset, which precociously secretes THBS1, creates a dysregulated microenvironment that inhibits the proliferation and function of the CD47^{lo} MuSC subset, impairing muscle regeneration.

FIG. 10. The fraction of CD47+ MuSCs increases in dystrophic mice. CD47 protein expression in dystrophic (Mdx-mTR G2 (G2)) and control (WT, mTR and Het) muscle stem cells was measured by CyTOF analysis at one and two months of age. The graph shows that the fraction of CD47+ stem cells significantly increases in dystrophic muscle stem cells. 2-way ANOVA analysis with Sidak correction for multiple comparisons was used to determine difference in the abundance of CD47+ cells between groups.

FIG. 11. Thrombospondin-1 blockade increases proliferation of dystrophic MuSCs. a₇integrin⁺/CD34⁺ MuSCs sorted from dystrophic G2 mice were cultured in growth media for 6 days on biomimetic hydrogels in the presence (+) or absence (−) of a blocking antibody to THBS1 and changes in proliferation was monitored by microscopy analysis. Thrombospondin-1 blockade led to increased proliferation of G2 MuSCs.

### DETAILED DESCRIPTION

### 1. Introduction

The present disclosure provides methods and compositions for enhancing the mass, strength, function, maintenance, regeneration, and other properties of aged, atrophied, or dystrophic skeletal muscle. The disclosure is based, in part, on the discovery of discrete subpopulations of muscle stem cells (MuSCs) in aging, atrophied, and dystrophic skeletal muscle with decreased proliferative, regenerative, and functional properties. Such dysfunctional MuSCs show increased surface expression of CD47, a receptor for thrombospondin-1, and the deficiencies of the dysfunctional MuSCs can be improved, reversed, reduced, or otherwise ameliorated through the inhibition of thrombospondin-1, e.g., using blocking antibodies or other molecules that prevent its interaction with CD47 and/or activation of CD47. The present disclosure therefore provides compositions and methods involving the inhibition of thrombospondin-1 as a therapeutic target in aged, atrophied, or dystrophic muscle to improve MuSC function and thereby improve muscle mass, function, and strength.

### 2. General

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“Sarcopenia” refers to a loss of muscle mass, strength, and/or physical performance in association with age. Sarcopenia is a progressive process that can occur at different rates in different individuals and there is no minimum age for a diagnosis. For example, a human can be considered to have sarcopenia for the purposes of the present invention if they are at least, e.g., 20, 25, 30, 35, 40, 45, 50, 55, 60, 65, 70, 75 years old or older.

“Aged muscle” or “aging muscle” refers to any muscle that shows or potentially shows any signs or features associated with increasing age or passage of time in developed muscle, including, e.g., loss of muscle mass or strength, decreased protein synthesis, accumulation of intra- and extra-myocellular lipids, mitochondrial dysfunction, expression of atrogenes (e.g., Atrogin1 and Murf), increased presence of senescent cells, increased levels of CD47 on the MuSC cell surface, decreased proliferation of MuSCs, and other features. In some embodiments, aged or aging muscle refers to muscles in a subject with sarcopenia.

“Muscle atrophy” or “atrophic muscle” refers to any loss or wasting of muscle tissue, e.g., any amount of decrease of muscle size, mass, or function, for any reason, e.g., in relation to a condition such as sarcopenia, diabetes, muscular dystrophy, sarcopenic obesity, neuropathy, cancer cachexia, or HIV cachexia, frailty, or muscle atrophy resulting from immobilization or disuse.

“Thrombospondin-1” or “THBS1” is a protein encoded by the human THBS1 gene that is involved in cell-cell and cell-matrix interactions and that has been shown to inhibit proliferation of endothelial cells. It is a glycoprotein that is a subunit of a homotrimeric protein. Thrombospondin-1 can interact with a number of proteins, including cell adhesion receptors such as CD47, and the scavenger receptor CD36. The NCBI Gene ID for the human thrombospondin-1 gene (THBS1) is 7057, and the UniProt Accession no. for the human protein is P07996.

“CD47” or “Cluster of Differentiation 47” is a transmembrane protein that belongs to the Immunoglobulin superfamily and is present, e.g., on the surface of MuSCs. CD47 (see, e.g., UniProtKB—A0A0A1TSG4) is encoded by the CD47 gene (see, e.g., NCBI Gene ID 961). CD47 has dual functions as ligand and receptor. For example, CD47 is a ligand for SIRPα through which CD47 prevents phagocytosis, and a receptor for the extracellular matrix protein THBS1. In addition, CD47 is also known as Integrin Associated Protein (IAP) because it interacts with integrins. While CD47 is expressed on all cells, including erythrocytes, its expression levels are transiently regulated in different contexts, modulating its function.

A “thrombospondin-1 inhibitor” refers to any agent that is capable of inhibiting, reducing, decreasing, attenuating, abolishing, eliminating, slowing, or counteracting in any way any aspect of the expression, stability, or activity of thrombospondin-1. A thrombospondin-1 inhibitor can, for example, reduce any aspect of the expression, e.g., transcription, RNA processing, RNA stability, or translation of a gene encoding thrombospondin-1, e.g., the human THBS/gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a thrombospondin-1 inhibitor can, for example, reduce the activity, e.g., CD47 binding activity, of thrombospondin-1 by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a thrombospondin-1 inhibitor can, for example, reduce the stability of a thrombospondin-1 enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “thrombospondin-1 inhibitor” can be any molecule, either naturally occurring or synthetic, e.g., antibody, antibody fragment, peptide, protein, oligopeptide (e.g., from about 5 to about 25 amino acids in length, e.g., about 5, 10, 15, 20, or 25 amino acids in length), small molecule (e.g., an organic molecule having a molecular weight of less than about 2500 daltons, e.g., less than 2000, less than 1000, or less than 500 daltons), polysaccharide, lipid, fatty acid, inhibitory RNA (e.g., siRNA, shRNA, microRNA, CRISPR gRNA), messenger RNA, modified RNA, polynucleotide, oligonucleotide, e.g. antisense oligonucleotide, aptamer, affimer, drug compound, or other compound.

The terms “expression” and “expressed” refer to the production of a transcriptional and/or translational product, e.g., of a nucleic acid sequence encoding a protein (e.g., thrombospondin-1). In some embodiments, the term refers to the production of a transcriptional and/or translational product encoded by a gene (e.g., the human THBS1 gene) or a portion thereof. The level of expression of a DNA molecule in a cell may be assessed on the basis of either the amount of corresponding mRNA that is present within the cell or the amount of protein encoded by that DNA produced by the cell.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a thrombospondin-1 protein). Examples of antibody-binding fragments include, but are not limited to, a Fab fragment (a monovalent fragment consisting of the VL, VH, CL, and CHI domains), F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen (see, e.g., FUNDAMENTAL IMMUNOLOGY (Paul ed., 4th ed. 2001).

The phrase “specifically binds” refers to a molecule (e.g., a thrombospondin-1 inhibitor such as a small molecule or antibody) that binds to a target with greater affinity, avidity, more readily, and/or with greater duration to that target in a sample than it binds to a non-target compound. In some embodiments, a molecule that specifically binds a target (e.g., thrombospondin-1) binds to the target with at least 2-fold greater affinity than non-target compounds, e.g., at least 3-fold, 4-fold, 5-fold, 6-fold, 7-fold, 8-fold, 9-fold, 10-fold, 20-fold, 25-fold, 50-fold or greater affinity. For example, in some embodiments, a molecule that specifically binds to thrombospondin-1 will typically bind to thrombospondin-1 with at least a 2-fold greater affinity than to a non-thrombospondin-1 target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer,” “administering,” or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. One skilled in the art will know of additional methods for administering a therapeutically effective amount of the compounds described herein for preventing or relieving one or more symptoms associated with a disease or condition.

The term “therapeutically effective amount” or “therapeutically effective dose” or “effective amount” refers to an amount of a compound (e.g., thrombospondin-1 inhibitor) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, ongoing disease process and type of treatment desired (e.g., aggressive vs. conventional treatment). Therapeutically effective amounts of a pharmaceutical compound or compositions, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutically acceptable carrier” refers to refers to a carrier or a diluent that does not cause significant irritation to an organism and does not abrogate the biological activity and properties of the administered compound.

The terms “subject,” “individual,” and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the invention, including nucleic acid inhibitors such as siRNA or shRNA, can be used with chemical modifications to enhance, e.g., stability and/or potency, e.g., as described in Dar et al. (2016) Scientific Reports 6: article no. 20031 (2016), and as presented in the database accessible at crdd.osdd.net/servers/simamod/.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

### 4. Methods of Enhancing Muscle Mass in Aged Muscles

The present disclosure provides methods of increasing the function, mass, strength, and other properties of aged muscle in a subject, e.g., a human subject, comprising administering a thrombospondin-1 inhibitor to the subject. The administration of the thrombospondin-1 inhibitors can be systemic or local, e.g., by intramuscular injection, and can enhance any of a number of aspects of the aged muscle, including enhancing mass, function, strength, or any other measure of muscle function in the subject. In particular embodiments, the administration of the thrombospondin-1 inhibitor leads to an increase in MuSC proliferation and activation in a muscle, and thereby to an increase in the regenerative potential of the muscle. In some embodiments, the aged, atrophied, or dystrophic skeletal muscle is uninjured. In some embodiments, the aged, atrophied, or dystrophic skeletal muscle is injured, and the thrombospondin-1 inhibitor accelerates recovery from the injury.

In one embodiment, provided herein are methods of enhancing a muscle function of an aged, atrophied, or dystrophic skeletal muscle in a subject, the method comprising: administering to the aged, atrophied, or dystrophic skeletal muscle a thrombospondin-1 inhibitor in an amount effective to inhibit the binding of thrombospondin-1 to CD47 and/or inhibit the activation of CD47 signaling by thrombospondin-1, thereby enhancing a muscle function of the aged, atrophied, or dystrophic skeletal muscle in the subject.

In another embodiment, provided herein are methods of increasing muscle mass, muscle strength, and/or muscle endurance of an aged, atrophied, or dystrophic skeletal muscle in a subject, the method comprising: administering to the aged, atrophied, or dystrophic skeletal muscle a thrombospondin-1 inhibitor in an amount effective to inhibit the binding of thrombospondin-1 to CD47 and/or inhibit the activation of CD47 signaling by thrombospondin-1, thereby increasing muscle mass, muscle strength, and/or muscle endurance of the aged, atrophied, or dystrophic skeletal muscle in the subject.

In another embodiment, a method of rejuvenating an aged, atrophied, or dystrophic skeletal muscle in a subject having one or more biomarkers of aging is provided, the method comprising: administering to the subject a thrombospondin-1 inhibitor in an amount effective to inhibit the binding of thrombospondin-1 to CD47 and/or inhibit the activation of CD47 signaling by thrombospondin-1, thereby rejuvenating the aged, atrophied, or dystrophic skeletal muscle in the subject.

The methods provided herein may be used to enhance a function of aged, atrophied, or dystrophic skeletal muscle. The methods provided herein may be used to rejuvenate aged, atrophied, or dystrophic skeletal muscle. The methods provided herein may be used to increase muscle mass, muscle strength, muscle force, and/or muscle endurance of aged, atrophied, or dystrophic skeletal muscle. The methods may be used to regenerate a population of muscle cells in a subject having a condition or disease associated with muscle damage, injury, or atrophy. The methods may be used for treating a condition or disease associated with muscle damage, injury or atrophy in a subject in need thereof. The methods may be used for treating muscle damage, muscle injury or muscle atrophy in a subject. The methods may be used for stimulating the proliferation of a population of isolated muscle cells, for example for ex vivo applications.

### Subjects

The subject can be any subject. e.g. a human or other mammal, with aged, atrophied, or dystrophic skeletal muscle, or at risk of having aged, atrophied, or dystrophic skeletal muscle. In some embodiments, the subject is a human. In particular embodiments, the subject is an adult (e.g., an adult with age-related sarcopenia). In some embodiments, the subject is a child (e.g., a child with a muscular dystrophy such as Duchenne muscular dystrophy). In some embodiments, the subject is female (e.g., an adult female). In some embodiments, the subject is male (e.g., an adult male).

In some embodiments, the subject is human, and the method further comprises a step in which the human is selected for treatment with the thrombospondin-1 inhibitor based on his or her age. For example, a human can be selected for treatment based on age who is over 20, 25, 30, 35, 40, 45, 50, 55, 60, 65, 70, 75, 80 years old or older, or any age in which the human has or potentially has sarcopenia or aged muscle. In some embodiments, the subject is determined to have aged muscle as determined using any method of assessing muscle strength or function, e.g., grip test, walk speed, muscle power test, functional tests, resistance tests, or treadmill, by imaging-based tests, by assessment of muscle mass, and/or by molecular or cellular analysis in, e.g., a muscle biopsy taken from the subject by a physician or other qualified medical professional.

In some embodiments, the method further comprises a step in which the human is selected for treatment with the thrombospondin-1 inhibitor based on a diagnosis of a condition involving atrophied or dystrophic skeletal muscle, e.g., spinal muscular atrophy, diabetes, frailty, muscular dystrophy, sarcopenic obesity, neuropathy, cancer cachexia, or HIV cachexia, or muscle atrophy resulting from immobilization or disuse. In some embodiments, the muscular dystrophy is selected from the group consisting of Duchenne muscular dystrophy, Becker muscular dystrophy, congenital muscular dystrophy, distal muscular dystrophy, Emery-Dreifuss muscular dystrophy, facioscapulohumeral muscular dystrophy, limb-girdle muscular dystrophy, myotonic muscular dystrophy, and oculopharyngeal muscular dystrophy.

In some embodiments, the skeletal muscle is uninjured. In some embodiments, the skeletal muscle is injured. The muscle can be any skeletal muscle of the body including, but not limited to, musculi pectoralis complex, latissimus dorsi, teres major and subscapularis, brachioradialis, biceps, brachialis, pronator quadratus, pronator teres, flexor carpi radialis, flexor carpi ulnaris, flexor digitorum superficialis, flexor digitorum profundus, flexor pollicis brevis, opponens pollicis, adductor pollicis, flexor pollicis brevis, iliopsoas, psoas, rectus abdominis, rectus femoris, gluteus maximus, gluteus medius, medial hamstrings, gastrocnemius, lateral hamstring, quadriceps mechanism, adductor longus, adductor brevis, adductor magnus, gastrocnemius medial, gastrocnemius lateral, soleus, tibialis posterior, tibialis anterior, flexor digitorum longus, flexor digitorum brevis, flexor hallucis longus, extensor hallucis longus, ocular muscles, pharyngeal muscles, sphincter muscles, hand muscles, arm muscles, foot muscles, leg muscles, chest muscles, stomach muscles, back muscles, buttock muscles, shoulder muscles, head and neck muscles, and the like.

In some embodiments, subjects are identified for treatment based on a diagnosis of sarcopenia or a potential for sarcopenia; based on a subject's age, i.e. an age associated with sarcopenia or of a potential for sarcopenia; or based on a detection of any of the herein-described features of aged muscle. For example, a detection in muscles of elevated levels of CD47 on the surface of MuSCs, of decreased MuSC proliferation or activation, of decreased muscle strength, mass, or function, etc., can indicate that the subject is a candidate for treatment with a thrombospondin-1 inhibitor. In some embodiments, the muscle has not been injured. In some embodiments, the muscle is uninjured.

In some embodiments, subjects are identified for treatment based on a diagnosis of a condition or disease associated with muscle atrophy; based on a determination of the presence of or potential for muscle atrophy, based on a subject's age, e.g., an age associated with sarcopenia or of a potential for sarcopenia, or based on a detection of any of the herein-described features of aged and/or atrophic muscle. For example, a detection in muscles of elevated levels of MuSCs with high surface levels of CD47, of decreased MuSC proliferation, of decreased protein synthesis in muscles, of decreased myofiber and/or myotube size, of decreased muscle mass, of decreased muscle strength, function or endurance, can indicate that the subject is a candidate for treatment with a thrombospondin-1 inhibitor.

In some embodiments, the subject is preparing to undergo surgery, is undergoing surgery, or has undergone surgery, and the methods and compositions are used in vivo to promote the regeneration of muscle tissue and thereby accelerate recovery from the surgery. In some embodiments, the subject is undergoing an ex vivo treatment involving the genetic correction of MuSCs, and the methods are compositions are used to expand the genetically modified MuSCs prior to transplantation. In some embodiments, the methods and compositions are used to expand autologous or heterologous MuSCs prior to their administration to a subject, i.e. as a cell therapeutic.

Subjects in need of muscle regeneration may have musculoskeletal injuries (e.g., fractures, strains, sprains, acute injuries, overuse injuries, and the like), post-trauma damages to limbs or face, athletic injuries, post-fractures in the aged, soft tissue hand injuries, muscle atrophy (e.g., loss of muscle mass), Duchenne muscular dystrophy (DMD), Becker muscular dystrophy, Fukuyama congenital muscular dystrophy (FCMD), limb-girdle muscular dystrophy (LGMD), congenital muscular dystrophy, facioscapulohumeral muscular dystrophy (FHMD), myotonic muscular dystrophy, oculopharyngeal muscular dystrophy, distal muscular dystrophy, Emery-Dreifuss muscular dystrophy, myotonia congenita, myotonic dystrophy, other muscular dystrophies, muscle wasting disease, such as cachexia due to cancer, end stage renal disease (ESRD), acquired immune deficiency syndrome (AIDS), or chronic obstructive pulmonary disease (COPD), post-surgical muscle weakness, post-traumatic muscle weakness, sarcopenia, inactivity (e.g., muscle disuse or immobility), urethral sphincter deficiency, urethral sphincter deficiency, neuromuscular disease, and the like.

Non-limiting examples of neuromuscular diseases include, but are not limited to, acid maltase deficiency, amyotrophic lateral sclerosis, Andersen-Tawil syndrome. Becker muscular dystrophy, Becker myotonia congenita. Bethlem myopathy, bulbospinal muscular atrophy, carnitine deficiency, carnitine palmityl transferase deficiency, central core disease, centronuclear myopathy, Charcot-Marie-Tooth disease, congenital muscular dystrophy, congenital myasthenic syndromes, congenital myotonic dystrophy, Cori disease, Debrancher enzyme deficiency, Dejerine-Sottas disease, dermatomyositis, distal muscular dystrophy, Duchenne muscular dystrophy, dystrophia myotonica. Emery-Dreifuss muscular dystrophy, endocrine myopathies, Eulenberg disease, facioscapulohumeral muscular dystrophy, tibial distal myopathy, Friedreich's ataxia, Fukuyuma congenital muscular dystrophy, glycogenosis type 10, glycogenosis type 11, glycogenosis type 2, glycogenosis type 3, glycogenosis type 5, glycogenosis type 7, glycogenosis type 9, Gowers-Laing distal myopathy, hereditary inclusion-body myositis, hyperthyroid myopathy, hypothyroid myopathy, inclusion-body myositis, inherited myopathies, integrin-deficient congenital muscular dystrophy, spinal-bulbar muscular atrophy, spinal muscular atrophy, lactate dehydrogenase deficiency, Lambert-Eaton myasthenic syndrome, McArdel disease, merosin-deficient congenital muscular dystrophy, metabolic diseases of muscle, mitochondrial myopathy, Miyoshi distal myopathy, motor neuron disease, muscle-eye-brain disease, myasthenia gravis, myoadenylate deaminase deficiency, myofibrillar myopathy, myophosphorylase deficiency, myotonia congenital, myotonic muscular dystrophy, myotubular myopathy, nemaline myopathy, Nonaka distal myopathy, oculopharyngeal muscular dystrophy, paramyotonia congenital, Pearson syndrome, periodic paralysis, phosphofructokinase deficiency, phosphoglycerate kinase deficiency, phosphoglycerate mutase deficiency, phosphorylase deficiency, polymyositis, Pompe disease, progressive external ophthalmoplegia, spinal muscular atrophy, Ullrich congenital muscular dystrophy, Welander distal myopathy, ZASP-related myopathy, and the like.

Muscle atrophy (e.g., muscle wasting) can be caused by or associated with, for example, normal aging (e.g., sarcopenia), genetic abnormalities (e.g., mutations or single nucleotide polymorphisms), poor nourishment, poor circulation, loss of hormonal support, disuse of the muscle due to lack of exercise (e.g., bedrest, immobilization of a limb in a cast, etc.), aging, damage to the nerve innervating the muscle, poliomyelitis, amyotrophic lateral sclerosis (ALS or Lou Gehrig's disease), heart failure, liver disease, diabetes, obesity, metabolic syndrome, demyelinating diseases (e.g., multiple sclerosis, Charcot-Marie-Tooth disease, Pelizaeus-Merzbacher disease, encephalomyelitis, neuromyelitis optica, adrenoleukodystrophy, and Guillian

The assessment of skeletal muscle function, strength, mass, or of any of the herein-described features in a subject can be assessed using any of a wide variety of methods known to those of skill in the art, e.g., by analysis of muscle performance such as by grip test, measurement of tetanic force, walk speed, muscle power test, functional tests, resistance tests, or treadmill, by imaging-based tests, by assessment of muscle mass, and/or by molecular or cellular analysis in, e.g., a muscle biopsy taken from the subject. In some embodiments, a muscle is injured, e.g., using notexin, and the effect of the inhibitor on the regeneration of the muscle is assessed, using any of the herein-described methods.

In some embodiments, the subject is a farm animal, e.g. livestock for human consumption, such as a porcine, bovine, ovine, poultry, or fish, and the methods are used, e.g., to enhance muscle mass, function, or strength in an aging animal, i.e., an animal with aged muscle. In some embodiments, a vector or expression cassette comprising a polynucleotide encoding a polypeptide inhibitor of thrombospondin-1, e.g., a blocking antibody or antibody fragment, is introduced into the animal such that the polypeptide inhibitor is expressed in the cells of the animal, e.g., the muscle cells. In some embodiments, the animal is administered a small molecule inhibitor of thrombospondin-1. In some embodiments, a vector or expression cassette comprising a nucleic acid inhibitor of thrombospondin-1, e.g., an shRNA, is introduced into the animal such that the nucleic acid inhibitor is expressed in the cells of the animal, e.g., the muscle cells. In some embodiments, gene therapy is used, e.g., such that all or part of an endogenous thrombospondin-1 encoding gene is replaced with a form of the gene that is less active, less stable, or less highly expressed in cells, e.g., MuSCs, of the animal. In some embodiments, modified RNA, e.g., a chemically modified RNA inhibitor such as shRNA or a chemically modified mRNA encoding a polypeptide thrombospondin-1 inhibitor is introduced into the animal such that the RNA inhibitor or expressed protein inhibitor is present in muscle cells of the animal.

### Thrombospondin-1 Inhibitors

Any agent that reduces, decreases, counteracts, attenuates, inhibits, blocks, downregulates, or eliminates in any way the expression, stability or activity (e.g., CD47-binding or activating ability) of thrombospondin-1 in aged, atrophied, or dystrophic skeletal muscle can be used in the present methods. Inhibitors can be antibodies, e.g., blocking antibodies or antibody fragments, small molecule compounds, peptides, polypeptides, nucleic acids, or any other molecule that reduces, decreases, counteracts, attenuates, inhibits, blocks, downregulates, or eliminates in any way the expression, stability and/or activity of thrombospondin-1, e.g., the ability of thrombospondin-1 to bind to and/or activate the CD47 receptor. In particular embodiments, the inhibitor is a blocking antibody or antibody fragment. e.g., blocking antibody A6.1, or a fragment thereof, available from Thermofisher scientific Cat #14-9756-82 and described in Annis et al. (2006) J Thromb Haemost. 2006 February; 4(2): 459-468, the entire disclosure of which is herein incorporated by reference.

In some embodiments, the inhibitor binds to thrombospondin-1. In some embodiments, the inhibitor does not bind to thrombospondin-1, but nevertheless the inhibitor prevent binding of thrombospondin-1 to CD47, or the activation of CD47 by thrombospondin-1.

In some embodiments, the thrombospondin-1 inhibitor decreases the activity (e.g., CD47 binding or activation), stability or expression of thrombospondin-1 by at least 10%, 15%, 20%, 25%, 30%, 35%, 40%, 45%, 50%, 55%, 60%, 65%, 70%, 75%, 80%, 85%, 90%, or more relative to a control level, e.g., a level determined in the absence of the inhibitor, in vivo or in vitro.

The efficacy of inhibitors can be assessed in any of a variety of ways, including in vitro and in vivo methods. For example, the efficacy can be assessed by measuring thrombospondin-1 binding to CD47, by measuring thrombospondin-1-mediated activation of CD47 as detected, e.g., through downstream signaling molecules such as cAMP, p38 MAPK, JAK/STAT, TGF-β. cAMP can be assessed, e.g., using a sensor such as a fluorescent cAMP downward sensor as described in the Examples. CD47 activation on MuSCs can also be assessed, e.g., by examining the proliferation of MuSCs using standard methods, e.g., quantifying cell numbers over time in vitro, or quantifying IdU incorporation in vitro or in vivo. The efficacy of an inhibitor can also be assessed in vivo, e.g., using a grip strength test or force measurement, with or without injury of the skeletal muscle being tested.

In some embodiments, the thrombospondin-1 inhibitor is considered effective if the binding and/or activation of CD47 using one of more of the herein-described methods is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a thrombospondin-1 inhibitor is considered effective if the level of expression of a thrombospondin-1-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

In some embodiments, the thrombospondin-1 inhibitor is considered effective if it improves one or more features of aged, atrophied, or dystrophic muscle, e.g., muscle mass, muscle strength (as detected, e.g., through grip test and force measurement assays), muscle regeneration ability (as detected, e.g., through transplantation assays), MuSC proliferation, MuSC activation, MuSC CD47 surface expression, MuSC Pax7 level (as detected, e.g., through CyTOF analysis), CD47 polyadenylation, CD47 3′ UTR length, or others. In some such embodiments, the inhibitor results in an increase in muscle mass and/or regeneration in the aged, atrophied, or dystrophic skeletal muscle relative to the level in the aged, atrophied, or dystrophic skeletal muscle prior to the administering of the thrombospondin-1 inhibitor, e.g., an increase of 5%, 10%, 20%, 30%, 40%, 50%, 60%, 70%, 80%, 90%, or more relative to the level in the aged, atrophied, or dystrophic skeletal muscle prior to the administering of the thrombospondin-1 inhibitor. In some embodiments, the inhibitor results in an increase in muscle mass and/or regeneration in the aged, atrophied, or dystrophic skeletal muscle to a level substantially similar to a level present in young and/or non-dystrophic skeletal muscle. In some such embodiments, the inhibitor results in an increase of at least 10% in muscle mass and/or regeneration in the aged, atrophied, or dystrophic skeletal muscle relative to the level in the aged, atrophied, or dystrophic skeletal muscle prior to the administering of the thrombospondin-1 inhibitor.

In some embodiments, the inhibitor results in an increase in MuSC proliferation and/or activity in the aged, atrophied, or dystrophic skeletal muscle relative to the level in the aged, atrophied, or dystrophic skeletal muscle prior to the administering of the thrombospondin-1 inhibitor, e.g., an increase of 5%, 10%, 20%, 30%, 40%, 50%, 60%, 70%, 80%, 90%, or more relative to the level in the aged, atrophied, or dystrophic skeletal muscle prior to the administering of the thrombospondin-1 inhibitor. In some embodiments, the inhibitor results in an increase in MuSC proliferation and/or activity in the aged, atrophied, or dystrophic skeletal muscle to a level substantially similar to a level present in young and/or non-dystrophic skeletal muscle. In some embodiments, the inhibitor results in an increase in MuSC proliferation and/or activity of at least 10% in the aged, atrophied, or dystrophic skeletal muscle relative to the level in the aged, atrophied, or dystrophic skeletal muscle prior to the administering of the thrombospondin-1 inhibitor.

In some embodiments, the inhibitor results in a decrease in MuSC surface levels of CD47 in the aged, atrophied, or dystrophic skeletal muscle relative to the level in the aged, atrophied, or dystrophic skeletal muscle prior to the administering of the thrombospondin-1 inhibitor. e.g., a decrease of 5%, 10%, 20%, 30%, 40%, 50%, 60%, 70%, 80%, 90%, or more relative to the level in the aged, atrophied, or dystrophic skeletal muscle prior to the administering of the thrombospondin-1 inhibitor. In some embodiments, the inhibitor results in a decrease in MuSC surface levels of CD47 in the aged, atrophied, or dystrophic skeletal muscle to a level substantially similar to a level present in young and/or non-dystrophic skeletal muscle. In some embodiments, the inhibitor results in a decrease in MuSC surface levels of CD47 in the aged, atrophied, or dystrophic skeletal muscle of at least 10% relative to the level in the aged, atrophied, or dystrophic skeletal muscle prior to the administering of the thrombospondin-1 inhibitor.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth. Integrative and Comparative Biology, 2014, 54:396-406; Thellin et al., Biotechnology Advances, 2009, 27:323-333, and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the thrombospondin-1 inhibitor is considered effective if the level of expression of a thrombospondin-1-encoding polynucleotide is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a thrombospondin-1 inhibitor is considered effective if the level of expression of a thrombospondin-1-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a thrombospondin-1 inhibitor can also be assessed by detecting protein expression or stability. e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA): immunoradiometric assays (IRMA); immunofluorescence (IF); fluorescence polanzation immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

For determining whether a thrombospondin-1 protein levels are decreased in the presence of a thrombospondin-1 inhibitor, the method comprises comparing the level of the protein (e.g., thrombospondin-1 protein) in the presence of the inhibitor to a reference value, e.g., the level in the absence of the inhibitor. In some embodiments, a thrombospondin-1 protein is decreased in the presence of an inhibitor if the level of the thrombospondin-1 protein is decreased by at least 10%, at least 20%, at least 30%, at least 40%, at least 50%, at least 60%, at least 70%, at least 80%, at least 90% or more as compared to the reference value. In some embodiments, a thrombospondin-1 protein is decreased in the presence of an inhibitor if the level of the thrombospondin-1 protein is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

### Antibodies

In particular embodiments, the inhibitor is an anti-thrombospondin-1 antibody or an antigen-binding fragment thereof. In some embodiments, the antibody is a blocking antibody (i.e., an antibody that binds to a target and directly interferes with the target's function, e.g., CD47-binding or activating activity of thrombospondin-1). In some embodiments, the antibody is a neutralizing antibody (i.e., an antibody that binds to a target and negates the downstream cellular effects of the target). In particular embodiments, the antibody binds to mouse or human thrombospondin-1.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In particular embodiments, the antibody is selected from, derived from, or is a fragment of an antibody selected from the group consisting of A6.1 (Thermofisher scientific Cat #14-9756-82; Annis et al.; 2006, J. Thromb. Haemost. 4(2):459-468), or an antibody that blocks the CD47-THBS1 interaction, including, but not limited to, B6H12 (Rogers et al. 2016, Kidney international, 90(2):334-347 DOI: 10.1016/j.kint.2016.03.034), MIAP410 (Kojima, Y., et al. (2016). “CD47-blocking antibodies restore phagocytosis and prevent atherosclerosis.” Nature. DOI: 10.1038/nature18935), B6H12.2 (Majeti et al, 2009, Cell, 138(2):286-99. doi: 10.1016/j.cell.2009.05.045), ab33852 (Nevitt C et al., 2016, Am J Physiol Heart Circ Physiol 310: H1842-H1850, DOI: 10.1152/ajpheart.00086.2016), or any of the anti-THBS1 or anti-CD47 antibodies (including human, rat, or mouse THBS1 or CD47), disclosed in any of these references (e.g., Annis et al. 2006; Rogers et al. 2016; Kojima et al. 2016; Majeti et al. 2009; Nevitt et al. 2016; U.S. Pat. Nos. 10,870,699, 10,844,124, Japanese Patent Application JP2021048858A) and including antibodies mAb133, MA-I, and D4.6 (Annis et al. 2006).

In some embodiments, an anti-thrombospondin-1 antibody comprises a heavy chain sequence or a portion thereof, and/or a light chain sequence or a portion thereof, of an antibody sequence disclosed herein. In some embodiments, an anti-thrombospondin-1 antibody comprises one or more complementarity determining regions (CDRs) of an anti-thrombospondin-1 antibody as disclosed herein. In some embodiments, an anti-thrombospondin-1 antibody is a nanobody, or single-domain antibody (sdAb), comprising a single monomeric variable antibody domain, e.g., a single VHH domain.

For preparing an antibody that binds to thrombospondin-1, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. m PEDS 23:311 (2010); and Chao et al., Nature Protocols, 1.755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670: WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-thrombospondin-1 antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-thrombospondin-1 antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS). BioLayer interferometry (e.g., Octet™ (FortéBio. Inc., Menlo Park, Calif.)), and western blot analysis.

### Small Molecules

In some embodiments of the invention, thrombospondin-1 is inhibited by the administration of a small molecule inhibitor. Any small molecule inhibitor can be used that reduces, e.g., by 10%, 15%, 20%, 25%, 30%, 35%, 40%, 45%, 50%, 55%, 60%, 65%, 70%, 75%, 80%, 85%, 90%, or more, the expression, stability or activity of thrombospondin-1 relative to a control, e.g., the expression, stability or activity in the absence of the inhibitor. In particular embodiments, small molecule inhibitors are used that can reduce or prevent the binding of thrombospondin-1 to CD47 in vitro or in vivo, or to the activation of CD47 signaling by thrombospondin-1 binding.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a thrombospondin-1 polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a thrombospondin-1-encoding polynucleotide sequence (e.g., the human THBS1 gene, Gene ID: 7057).

In some embodiments, the methods described herein comprise treating a subject, e.g., a subject with sarcopenia or aging muscle, using an shRNA or siRNA. A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject with aging muscle comprises administering to the subject a therapeutically effective amount of a modified RNA or a vector comprising a polynucleotide that encodes an shRNA or siRNA capable of hybridizing to a portion of a thrombospondin-1 mRNA. In some embodiments, the vector further comprises appropriate expression control elements known in the art, including, e.g., promoters (e.g., inducible promoters or tissue specific promoters), enhancers, and transcription terminators.

In some embodiments, the agent is a thrombospondin-1-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent is an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a thrombospondin-1 mRNA. In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., 95%, 90%, 85%, 80%, 75%, or 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies, 4^{th} edition by N. S. Templeton; Translating Gene Therapy to the Clinic: Techniques and Approaches, 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see, e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Peptides

In some embodiments, the inhibitor is a peptide, e.g, a peptide that binds to and/or inhibits the activity or stability of thrombospondin-1. In some embodiments, the inhibitor is a peptide that prevents or decreases binding of thrombospondin-1 to CD47, e.g., on the surface of MuSCs. In some embodiments, the inhibitor is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and Modified RNA

In some embodiments, polynucleotides providing thrombospondin-1 inhibiting activity, e.g., a nucleic acid inhibitor such as an siRNA or shRNA, or a polynucleotide encoding a polypeptide that inhibits thrombospondin-1 such as a blocking antibody fragment, are introduced into cells, e.g., muscle cells, using an appropriate vector. Examples of delivery vectors that may be used with the present disclosure are viral vectors, plasmids, exosomes, liposomes, bacterial vectors, or nanoparticles. In some embodiments, any of the herein-described thrombospondin-1 inhibitors, e.g., a nucleic acid inhibitor or a polynucleotide encoding a polypeptide inhibitor, are introduced into cells, e.g., muscle cells, using vectors such as viral vectors. Suitable viral vectors include but not limited to adeno-associated viruses (AAVs), adenoviruses, and lentiviruses. In some embodiments, a thrombospondin-1 inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in muscle cells.

In some embodiments, the nucleic acid or protein inhibitors of thrombospondin-1 are introduced into a subject, e.g., into the muscles of a subject, using modified RNA. Various modifications of RNA are known in the art to enhance. e.g., the translation, potency and/or stability of RNA, e.g., shRNA or mRNA encoding a thrombospondin-1 polypeptide inhibitor, when introduced into cells of a subject. In particular embodiments, modified mRNA (mmRNA) is used, e.g., mmRNA encoding a polypeptide inhibitor of thrombospondin-1. In other embodiments, modified RNA comprising an RNA inhibitor of thrombospondin-1 expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/simamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used. See, e.g., Youn et al. (2015) Expert Opin Biol Ther, September 2; 15(9): 1337-1348; Kaczmarek et al. (2017) Genome Medicine 9:60; Gan et al. (2019) Nature comm. 10: 871; Chien et al. (2015) Cold Spring Harb Perspect Med. 2015; 5:a014035; the entire disclosures of each of which are herein incorporated by reference.

### 5. Methods of Administration

The compounds of the present invention can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In particular embodiments, the compounds are administered intramuscularly, e.g., by intramuscular injection. In particular embodiments, the compounds are administered intramuscularly. i.e. injected directly into the aged, atrophied, or dystrophic muscle.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period. In some embodiments, the inhibitor can be administered 1, 2, 3, 4, 5, 6, 7, 8, 9, 10 or more times.

Administration of the compound into a subject can be accomplished by methods generally used in the art. The quantity of the compound introduced will take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the condition or disease, including reducing or eliminating one or more symptoms or manifestations of the condition or disease.

Any number of skeletal muscles of the body may be directly injected with or otherwise administered the compounds of the present invention, such as, for example, the biceps muscle; the triceps muscle; the brachioradialus muscle; the brachialis muscle (brachialis anticus); the superficial compartment wrist flexors; the deltoid muscle; the biceps femoris, the gracilis, the semitendinosus and the semimembranosus muscles of the hamstrings; the rectus femoris, vastus lateralis, vastus medialis and vastus intermedius muscles of the quadriceps; the gastrocnemius (lateral and medial), tibialis anterior, and the soleus muscles of the calves; the pectoralis major and the pectoralis minor muscles of the chest; the latissimus dorsi muscle of the upper back; the rhomboids (major and minor); the trapezius muscles that span the neck, shoulders and back; the rectus abdominis muscles of the abdomen; the gluteus maximus, gluteus medius and gluteus minimus muscles of the buttocks; muscles of the hand; sphincter muscles; ocular muscles; and pharyngeal muscles.

### 6. Pharmaceutical Compositions

The pharmaceutical compositions of the compounds of the present invention may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see. e.g., REMINGTON′S PHARMACEUTICAL SCIENCES, 18TH ED., Mack Publishing Co., Easton, Pa. (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions of the invention are administered in a manner compatible with the dosage formulation, and in such amount as will be therapeutically effective. The quantity to be administered depends on a variety of factors including, e.g., the age, body weight, physical activity, hereditary characteristics, general health, sex and diet of the individual, the condition or disease to be treated, the mode and time of administration, rate of excretion, drug combination, the stage or severity of the condition or disease, etc. In certain embodiments, the size of the dose may also be determined by the existence, nature, and extent of any adverse side effects that accompany the administration of a therapeutic agent(s) in a particular individual.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g. an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

Methods for preparing such dosage forms are known to those skilled in the art (see. e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see. e.g., REMINGTON′S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side effects caused by induction of the immune response, etc. In some embodiments, for example, the thrombospondin-1 inhibitors of the invention are administered together with another compound aiming to enhance muscle mass, strength, or function.

### 7. Kits

Other embodiments of the compositions described herein are kits comprising a thrombospondin-1 inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of aging muscle. In some embodiments, the kit comprises an agent that antagonizes the expression or activity of thrombospondin-1. In some embodiments, the kit comprises an inhibitory nucleic acid (e.g., an antisense RNA, small interfering RNA (siRNA), microRNA (miRNA), short hairpin RNA (shRNA)), or a polynucleotide encoding a thrombospondin-1 inhibiting polypeptide, that inhibits or suppresses thrombospondin-1 mRNA or protein expression or activity, e.g., CD47-binding activity. In some embodiments, the kit comprises a modified RNA, e.g., a modified shRNA or siRNA, or a modified mRNA encoding a polypeptide thrombospondin-1 inhibitor. In some embodiments, the kit further comprises one or more plasmid, bacterial or viral vectors for expression of the inhibitory nucleic acid or polynucleotide encoding a thrombospondin-1-inhibiting polypeptide. In some embodiments, the kit comprises an antisense oligonucleotide capable of hybridizing to a portion of a thrombospondin-1-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a thrombospondin-1 protein. In some embodiments, the kit comprises a blocking peptide. In some embodiments, the kit comprises an aptamer (e.g., a peptide or nucleic acid aptamer). In some embodiments, the kit comprises an affirmer. In some embodiments, the kit comprises a modified RNA. In some embodiments, the kit comprises a small molecule inhibitor that binds to thrombospondin-1 and prevents or reduces its binding to and/or activation of CD47 activity. In some embodiments, the kit further comprises one or more additional therapeutic agents, e.g., agents for administering in combination therapy with the agent that antagonizes the expression or activity of thrombospondin-1.

In some embodiments, the kits can further comprise instructional materials containing directions (i.e., protocols) for the practice of the methods of this invention (e.g., instructions for using the kit for enhancing mass, strength, or function in aged muscle). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### 8. EXAMPLES

The following examples are offered to illustrate, but not to limit the claimed invention.

### Example 1: Aberrant CD47 Signaling by Dysfunctional Stem Cell Subsets Hinders the Regenerative Response in Aged Muscle

a) Introduction

Here we capitalize on multiparametric single-cell mass cytometry (25-27), which allows discovery of novel cell subsets within rare stem cell populations, to determine whether alterations in the relative abundance of MuSC subsets or in the regulation of their signaling networks is responsible for the decline of skeletal MuSC function in the course of aging. High dimensional single-cell analysis of MuSCs enabled us to identify two functionally and molecularly distinct subsets, defined by differential cell surface expression of CD47, with a relative abundance that is altered with aging.

We report that a shift in alternative polyadenylation choice dictates stem cell fate by increasing CD47 surface expression during the transition from stem to progenitor cells and that during aging this process is aberrant. CD47, known as the “don't eat me signal” on cancer cells (28), is also a receptor for thrombospondin-1, a cellular matrix protein shown by others to inhibit proliferation of endothelial cells (29-31), although a role in MuSCs or aging has not previously been described. We show that thrombospondin-1/CD47 signaling comprises a key negative feedback loop that regulates MuSC expansion during regeneration and is dysregulated in aged MuSCs. Notably, treatment with a monoclonal antibody to thrombospondin-1 that blocks its interaction with CD47 (32) alleviates the proliferative defect of aged MuSCs, and restores their regenerative potential in vivo to a level similar to that of young MuSCs. This study uncovers an unexpected role for thrombospondin-1 in a paracrine loop that hinders MuSC function in aging and identifies a means to surmount aberrant thrombospondin-1 signaling via immune blockade, leading to enhanced regeneration, findings with broad implications for sarcopenia and aged stem cells in other tissues.

b) Results

### CD47 Expression Levels Distinguish Functionally and Molecularly Distinct Aged Muscle Stem Cell Subsets.

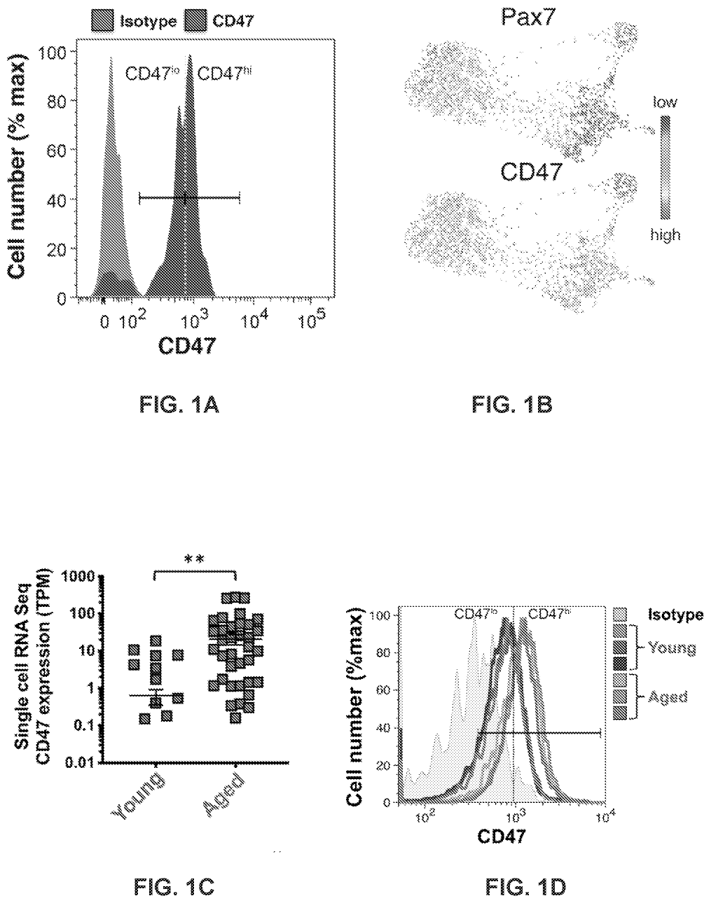

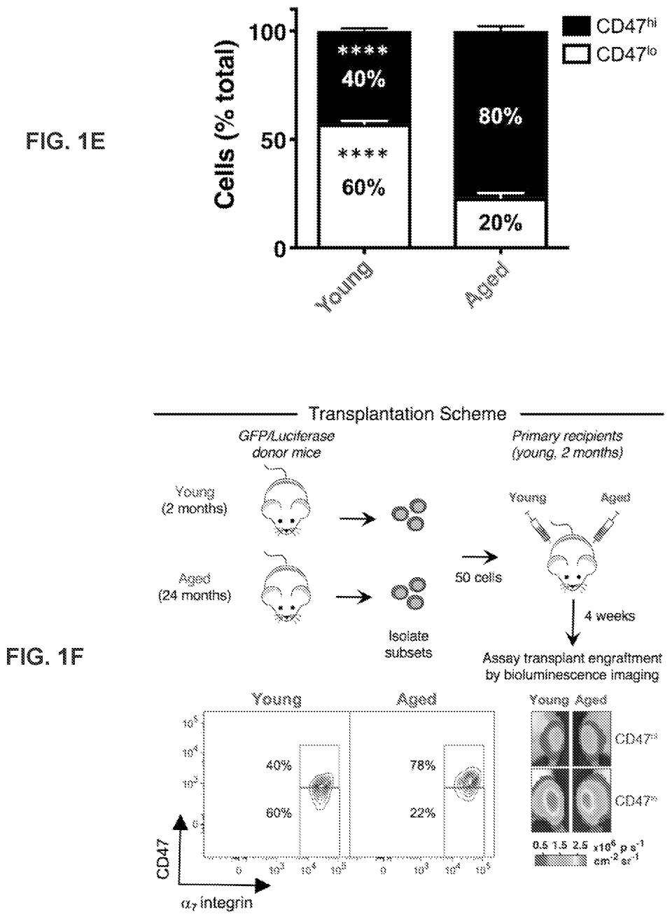

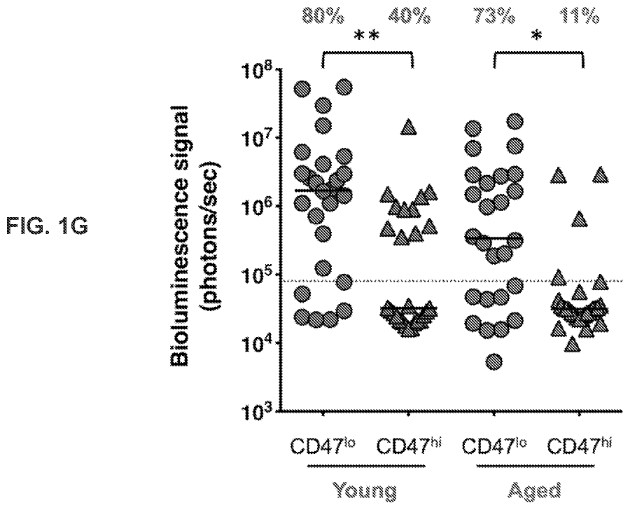

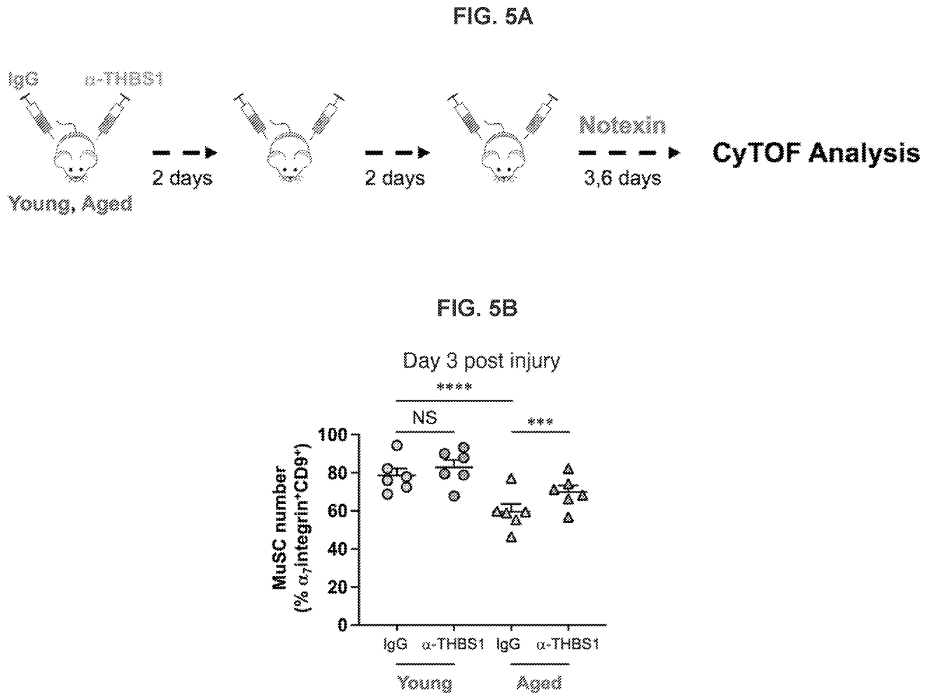

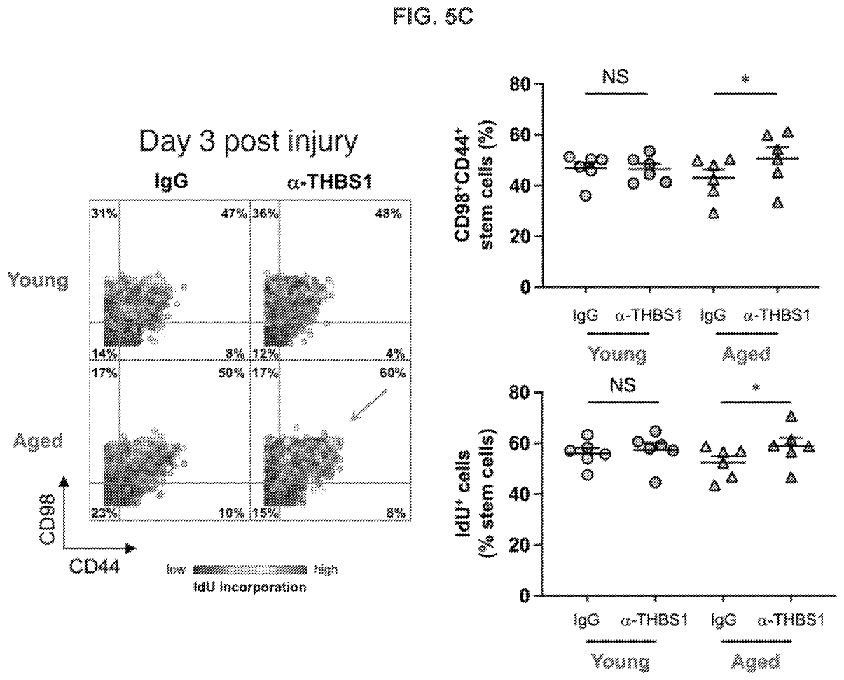

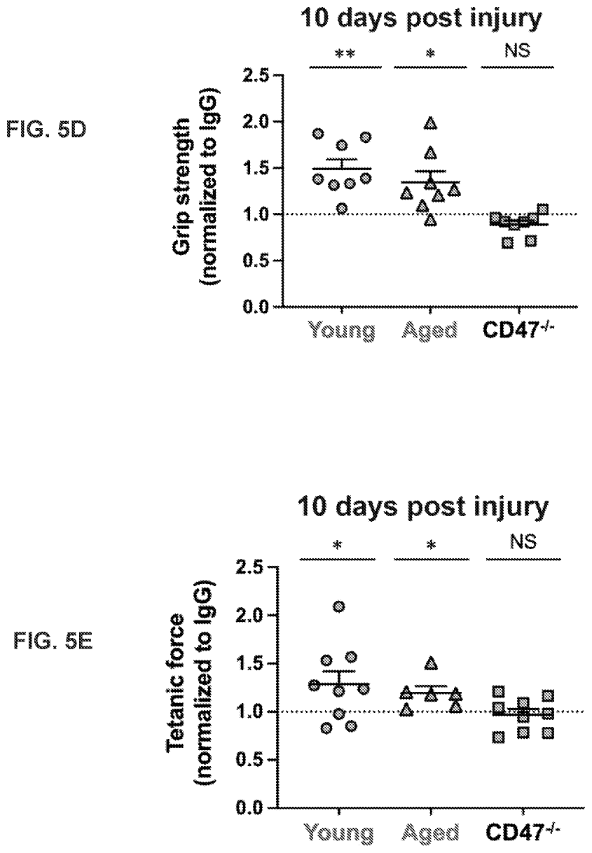

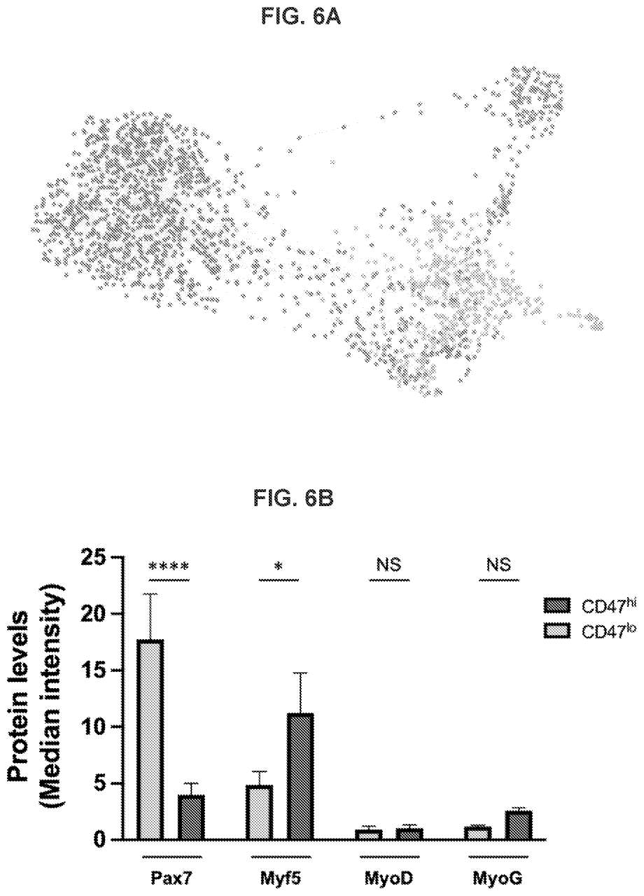

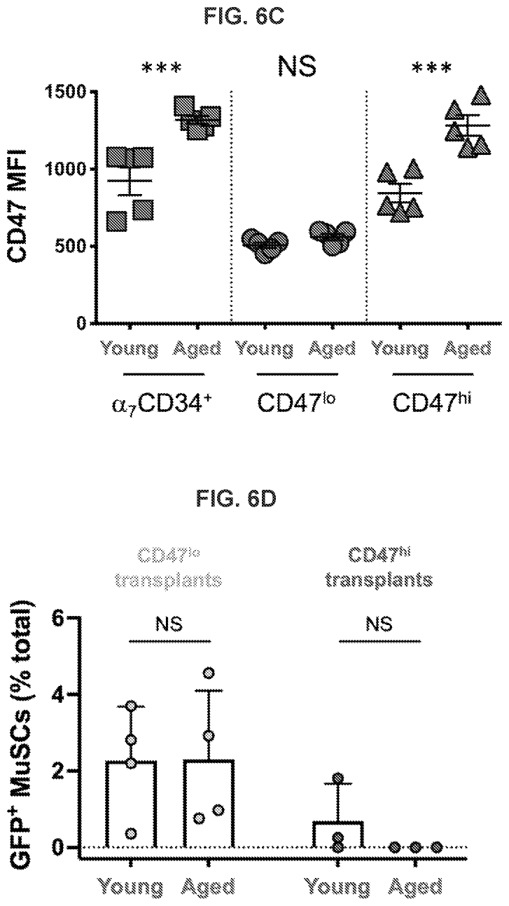

To identify cell surface markers that could distinguish novel MuSC subsets whose relative proportion is altered in the course of aging, we interrogated our previously described cell surface marker screen of single muscle cells²⁶. We observed that CD47 distinguished two subsets in young MuSCs (Lin⁻/α₇integrin⁺CD34⁺), CD47^{lo} and CD47^{hi} (FIG. 1A). We capitalized on our previous identification of muscle stem and progenitor markers (CD9, CD104), as well as activated MuSC markers (CD44, CD98) and included a CD47 antibody in our previously established CyTOF panel²⁶, with the goal of resolving MuSC heterogeneity in aging. Analysis of the high-dimensional CyTOF data, using X-shift clustering paired with single-cell force directed layout visualization, identified two distinct clusters distinguished by differential expression of CD47 and the transcription factor Pax7 (FIG. 1B). The cluster with intermediate to high expression levels of CD47 co-expressed low levels of Pax7 (FIG. 1B, right), whereas the cluster expressing low levels of CD47 co-expressed high levels of Pax7 (FIG. 1B, left). Single-cell RNA-seq analysis of young and aged MuSCs revealed a marked increase in CD47 mRNA expression during aging (FIG. 1C). Flow cytometry analysis of CD47 protein expression in single cells derived from young and aged muscle confirmed the presence of two MuSC subsets, CD47^{lo} and CD47^{hi} (FIG. 1D). We found that the proportion of CD47^{hi} MuSCs (FIGS. 1D, 1E) and the CD47 signal intensity (FIGS. 1D, 5A) increased with aging. These data demonstrate the presence of a previously unrecognized MuSC subset that is enriched in aged compared to young muscles.

To assess their regenerative potential, CD47^{lo} and CD47^{hi} MuSC subsets were isolated from young and aged GFP/Luciferase mice and transplanted into the irradiated Tibialis Anterior (TA) muscles of NOD/SCID mice. At 4 weeks post-transplant, the contribution of the donor cells to regenerated damaged tissues was determined by bioluminescence imaging (BLI) (FIG. 1F). Strikingly, based on the engrafted transplants, the CD47^{lo} subset isolated from young and aged donor mice exhibited the highest regenerative potential as shown by high engraftment frequencies and BLI signal intensity (FIGS. 1F, 1G). This finding suggests that elevated CD47 may play a role in the reduced engraftment seen in the unfractionated aged MuSC population²⁰.

### Alternative Polyadenylation Regulates CD47 Expression at the Onset of Myogenic Differentiation and is Altered in Aged Muscle Stem Cells.

In order to understand the mechanism underlying the accumulation of the CD47^{hi} subset with aging, we investigated the post-transcriptional regulation of CD47 expression. CD47 has been reported to undergo alternative polyadenylation in human cell lines, leading to differential subcellular localization^{33,34}. The short 3′ UTR traffics CD47 protein primarily to the endoplasmic reticulum, whereas the long 3′ UTR, which binds a complex containing the RNA binding protein HuR, progresses to the cell surface³³. HuR has been shown to increase at the onset of myogenic differentiation³⁵⁻³⁷. Hence, we hypothesized that the long 3′UTR isoform of the CD47 transcript was preferentially expressed during myogenic differentiation, resulting in CD47 protein localization at the cell surface. This hypothesis could explain the role of differential surface expression of CD47 protein in MuSC subsets from young and aged mice.

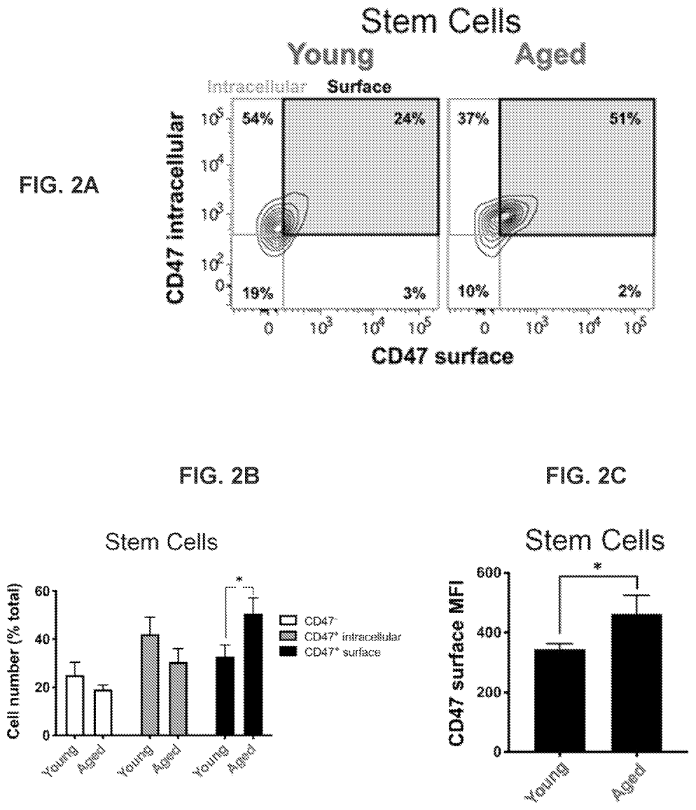

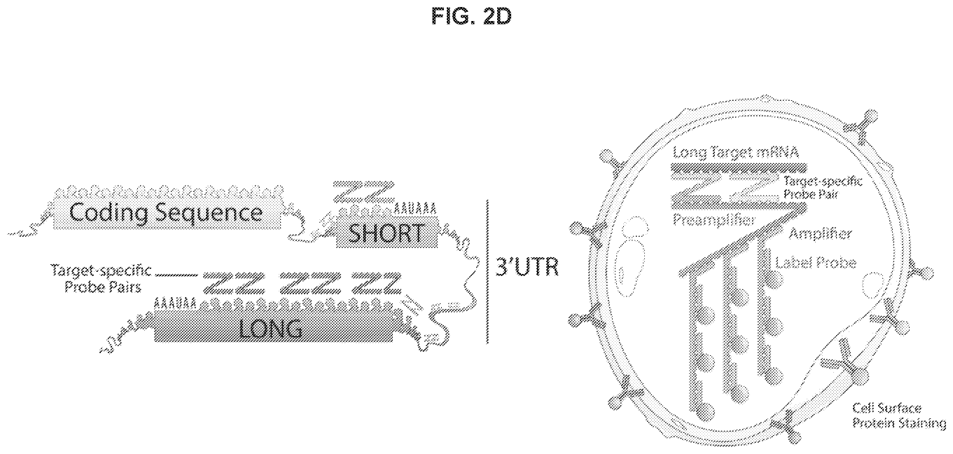

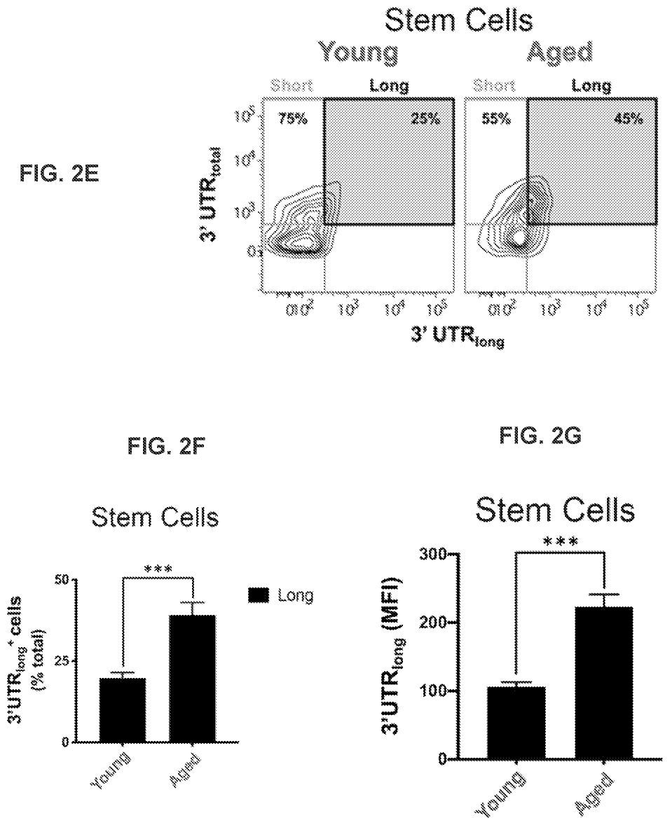

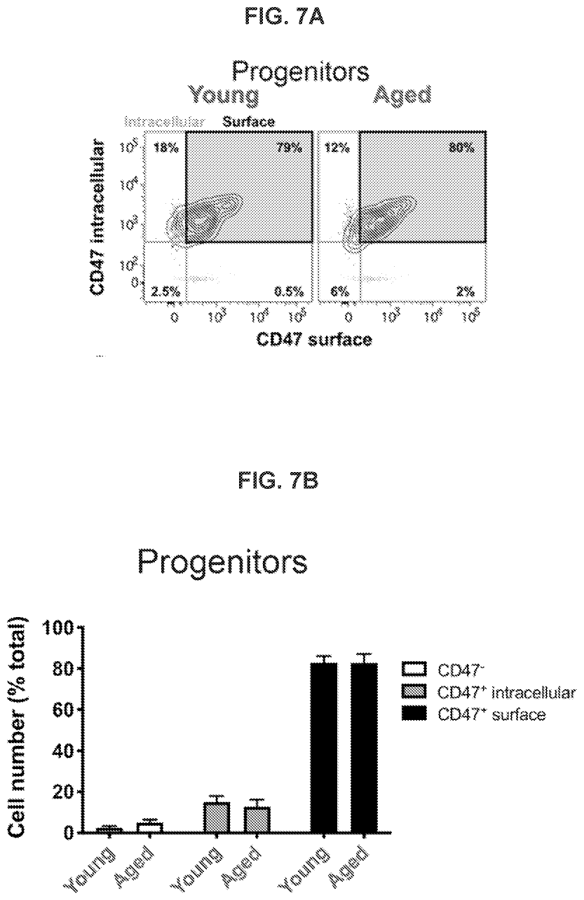

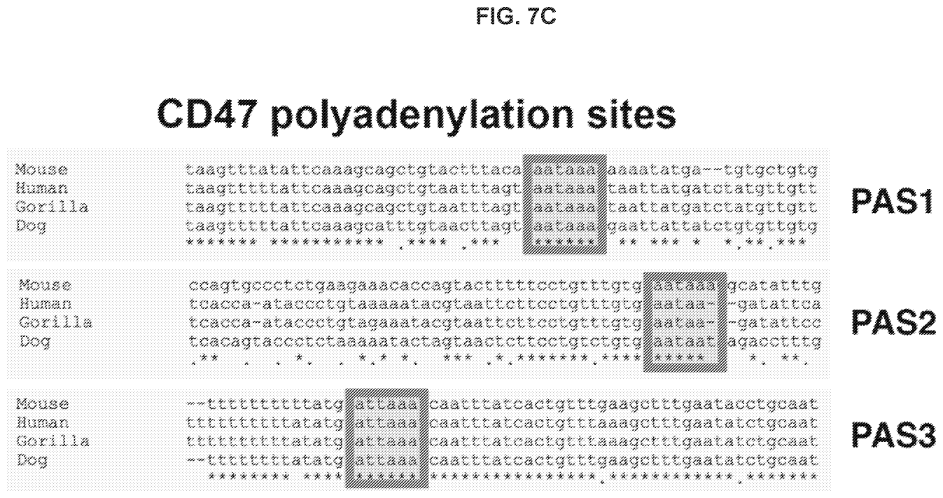

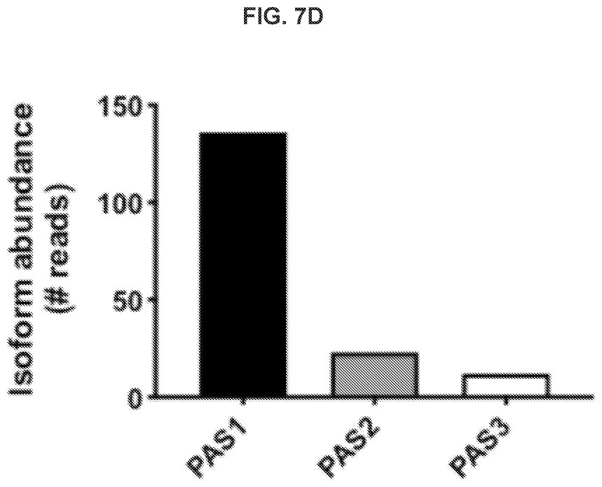

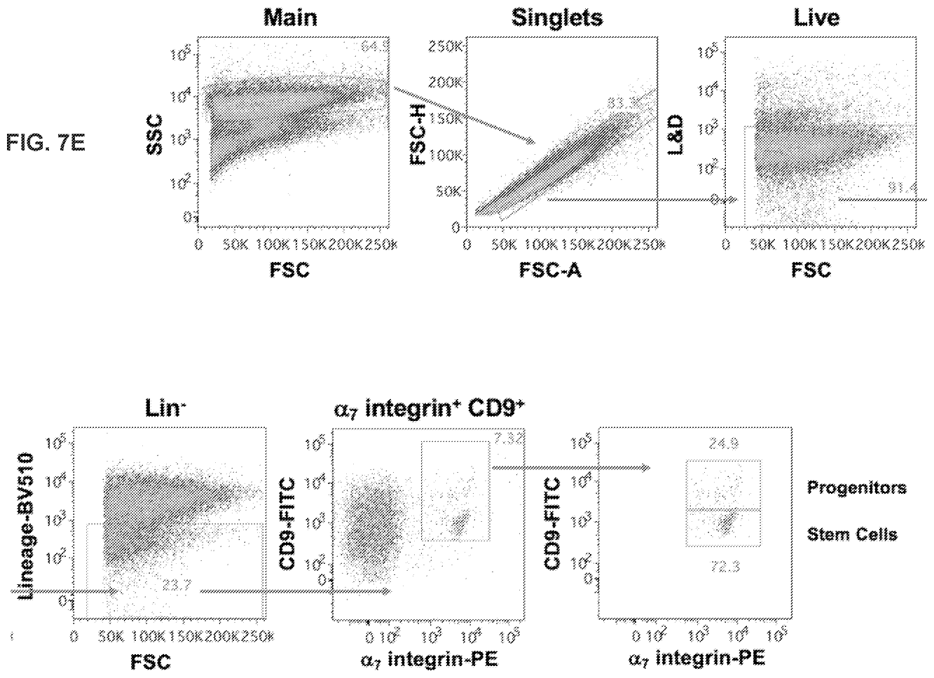

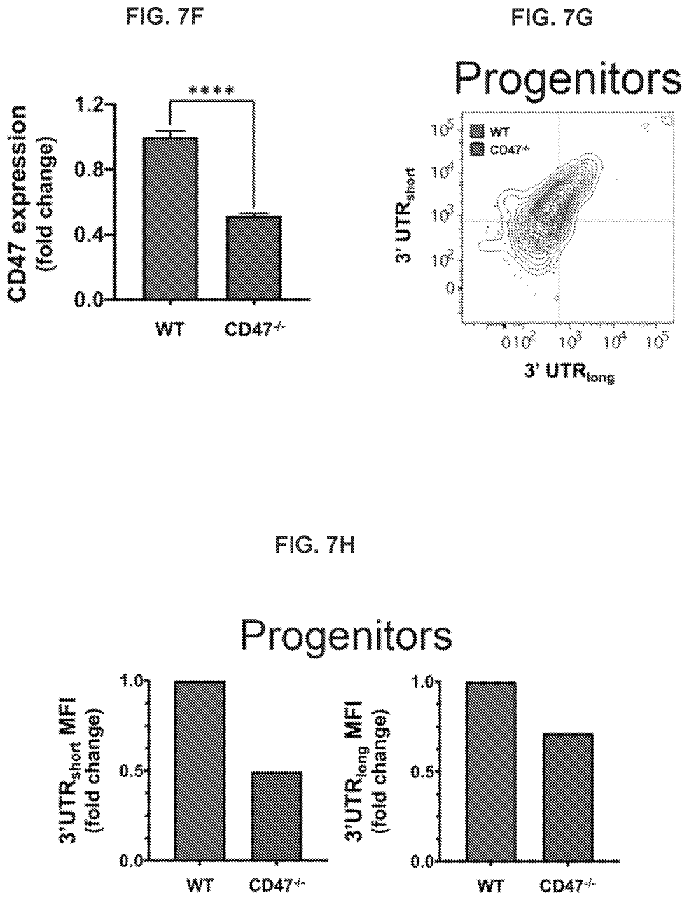

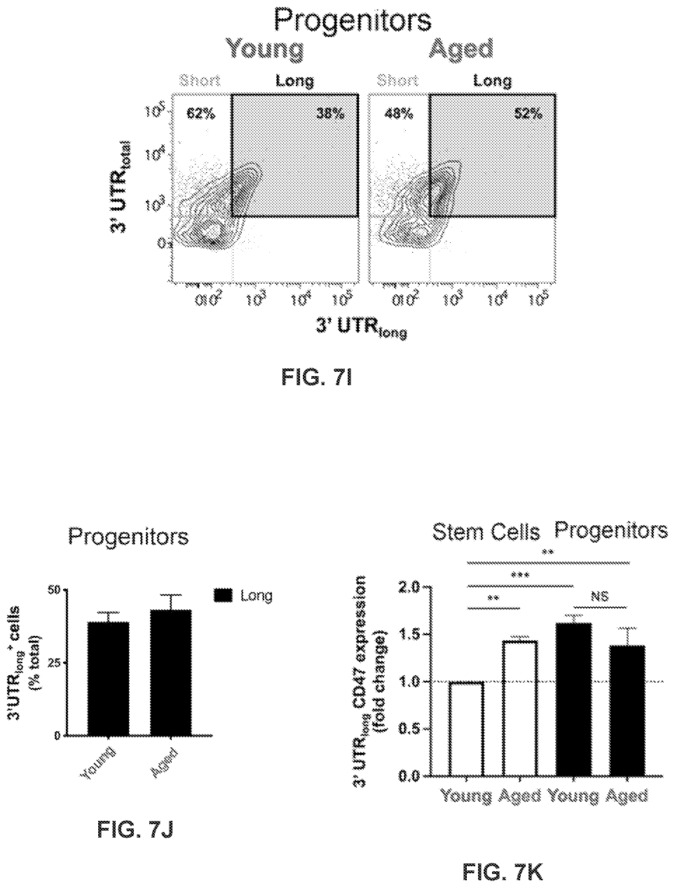

To assess whether stem and progenitor cells exhibited differential localization of CD47 protein, we measured its expression on both the cell surface and intracellularly by flow cytometry. Specifically, we performed surface staining followed by cell permeabilization and intracellular staining using the same CD47 antibody labeled with different fluorophores. We found that in young mice MuSCs primarily expressed CD47 intracellularly (FIGS. 2A, 2B), whereas muscle progenitor cells primarily expressed CD47 on the cell surface (FIGS. 7A-7B). In aged mice, the proportion of MuSCs expressing surface CD47 was significantly greater than in young mice (FIGS. 2A, 2B), whereas the proportion of muscle progenitor cells expressing surface CD47 was comparable in young and aged mice (FIGS. 7A-7B). In addition. MuSCs from aged mice exhibited higher expression levels of surface CD47 protein compared to MuSCs from young mice (FIG. 2C).

To map and determine the abundance of the different 3′UTR isoforms of murine CD47 mRNA we performed transcript alignment from different species (FIG. 7C) in combination with analysis of published datasets obtained by 3′ region extraction and deep sequencing of murine cell lines³⁸. We identified three CD47 mRNA isoforms with 3′UTR of different lengths that showed, from shortest to longest, progressively lower usage (FIG. 7D). We focused our analysis on the most prevalent isoforms containing the polyadenylation sites, PAS1 or PAS2, herein referred to as short and long, respectively (FIG. 2D).

To measure the distribution of short and long 3′UTR transcripts at the single cell level, we used branched DNA technology³⁹ to specifically label individual isoforms in single cells with target-specific probes spanning 600-800 bases upstream of each polyadenylation site (FIG. 2D). We stained single-cell suspensions of muscle cells from either young or aged mice, first with antibodies against the surface markers α₇integrin and CD9, to distinguish both stem (Lin⁻/α₇ integrin⁺/CD9^{int}) and progenitor (Lin⁻/α₇ integrin⁺/CD9^{hi}) cells (FIG. 7E), and then intracellularly with probes specific to the different isoforms of the CD47 mRNA (FIG. 2D). To determine probe specificity we included samples from CD47^{-/-} mice⁴⁰, which expressed reduced levels of both total CD47 mRNA by qPCR and of the individual mRNA isoforms by single-cell analysis (FIGS. 7F-7H).

In young mice, a smaller proportion of stem cells (FIGS. 2E, 2F) expressed the CD47 mRNA isoform with long 3′UTR (3′UTR_{long}) compared to the progenitor cells (FIGS. 7F, 7G). In aged mice, the proportion of cells and levels of expression of CD47 mRNA isoform with long 3′UTR was comparable in both stem and progenitor cells (FIGS. 2E, 2F; FIGS. 7I, 7J). Moreover, the expression level of the CD47 mRNA isoform with long 3′UTR was higher in aged compared to young stem cells (FIG. 2G). We confirmed these findings by RT-PCR of sorted muscle stem and progenitor cells (FIG. 7K). These results suggest that alternative polyadenylation modulates surface expression of CD47 at the onset of myogenic differentiation and determines the increased surface expression of CD47 in aged MuSCs.

### Aberrant Thrombospondin-1 Signaling Via CD47 Inhibits the Proliferative Capacity of Aged Muscle Stem Cells.

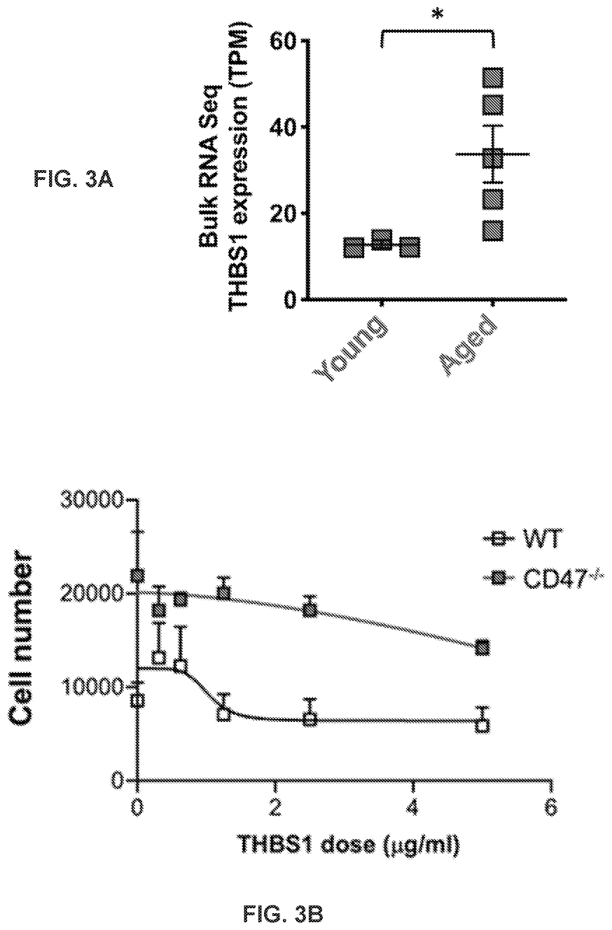

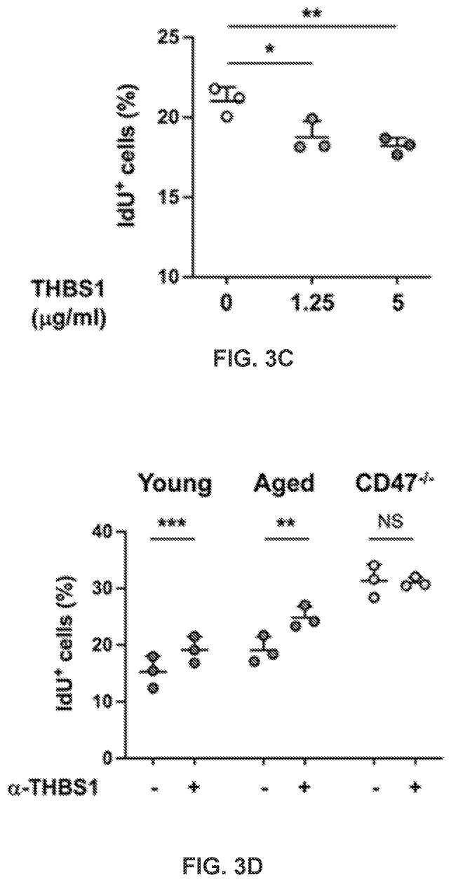

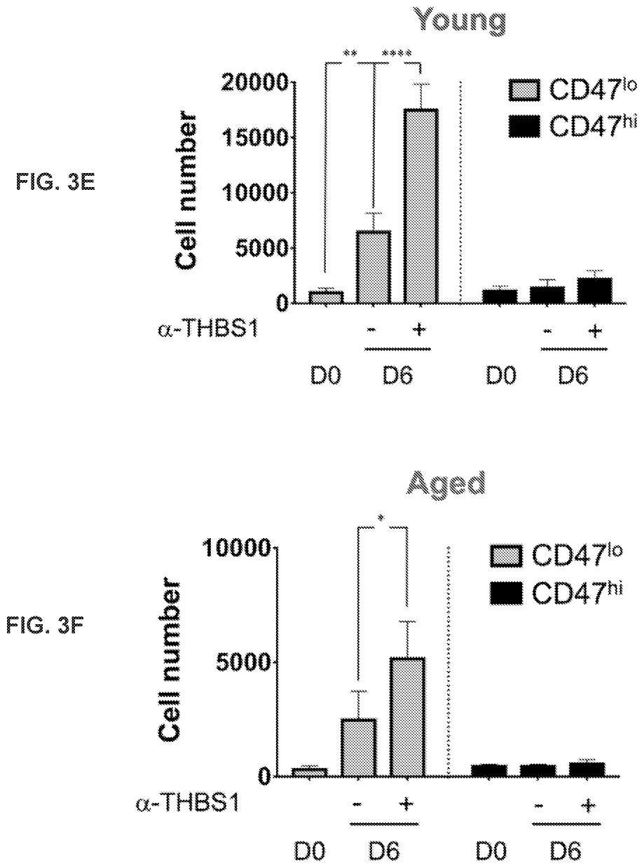

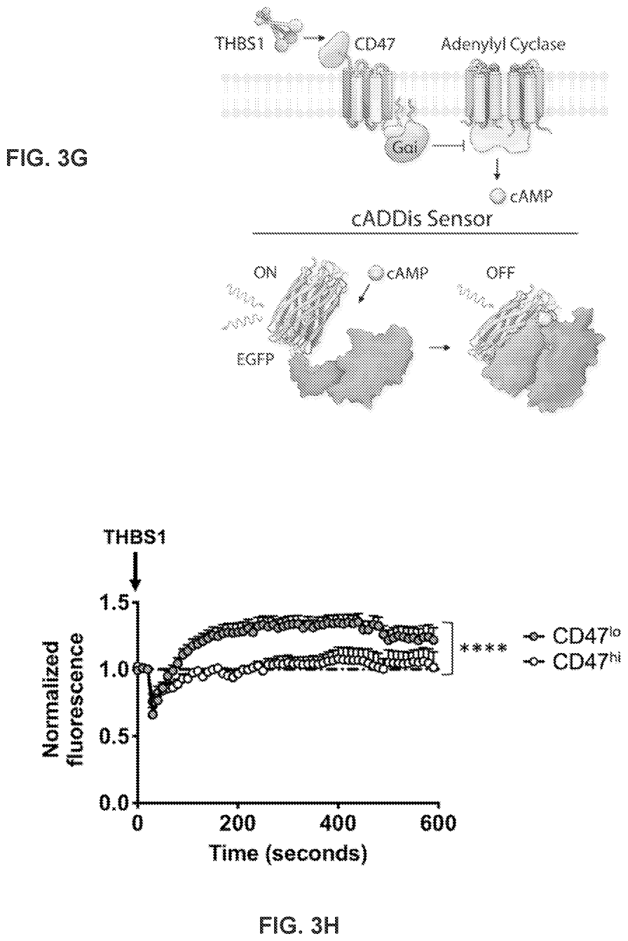

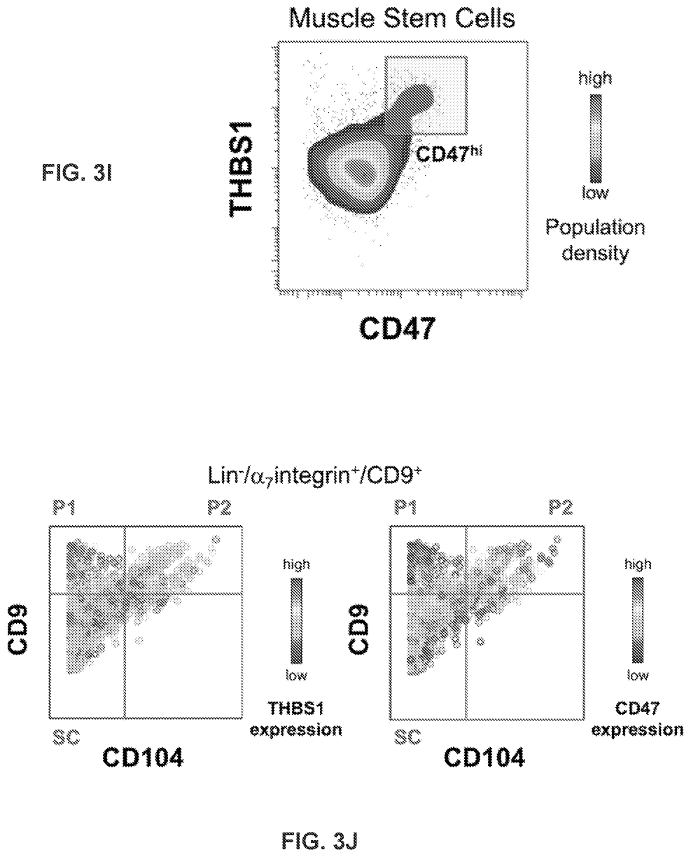

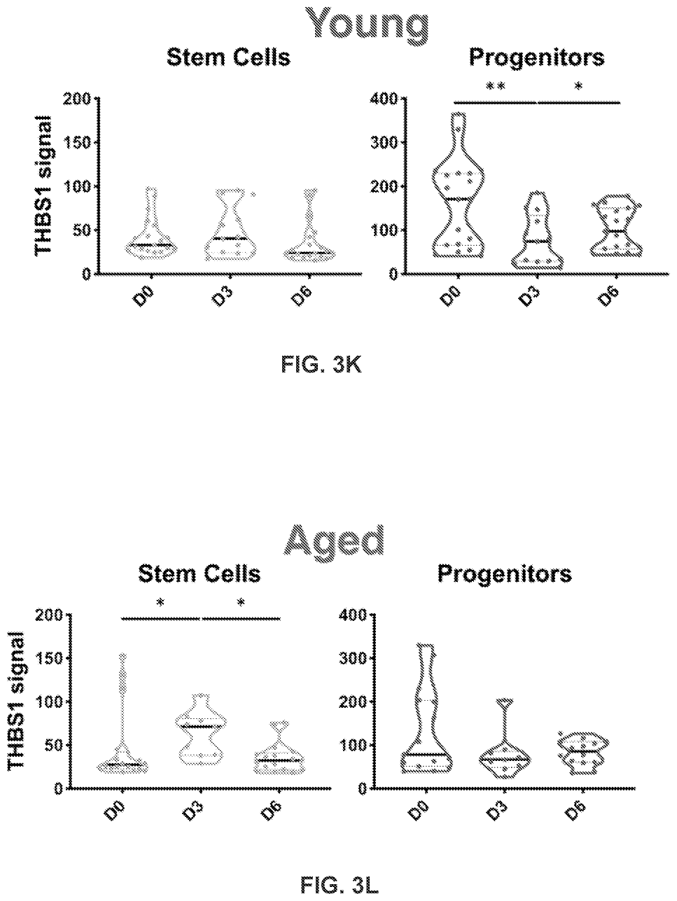

CD47 has dual functions as ligand and receptor⁴¹. Also known as the “don't eat me signal” on neoplastic cells^{28,42}, CD47 is a ligand for SIRP-α, a receptor present on the surface of immune cells, which upon binding to CD47 delivers an inhibitory signal that blocks phagocytosis²⁸. In addition, CD47 is known as Integrin Associated Protein (IAP) because it interacts with integrins⁴³. Finally, CD47 also serves as a receptor for thrombospondin-1, a cellular matrix protein that has been shown to inhibit the proliferation of endothelial cells^{29-31,41} We found that thrombospondin-1 transcript expression was increased in aged compared to young MuSCs (FIG. 3A). To establish whether increased thrombospondin-1 expression could account for the reduced proliferative potential of aged MuSCs, we investigated the thrombospondin-1/CD47 signaling axis and performed a series of in vitro and in vivo studies.

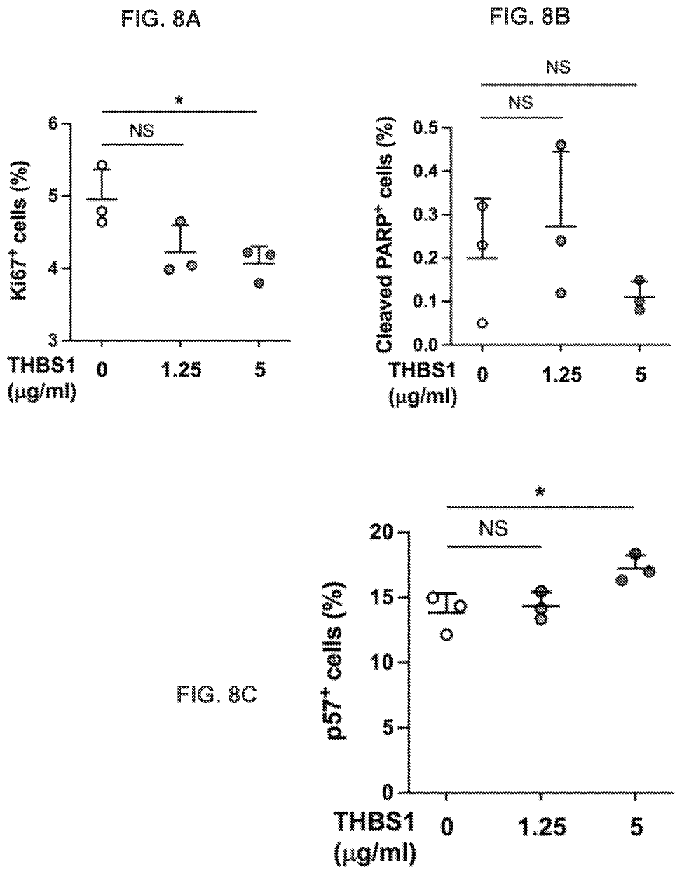

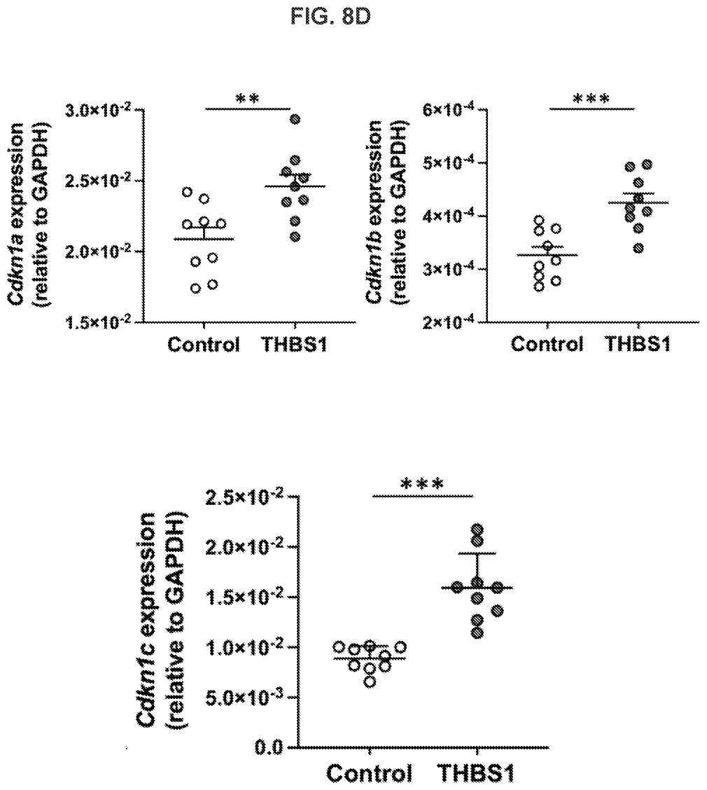

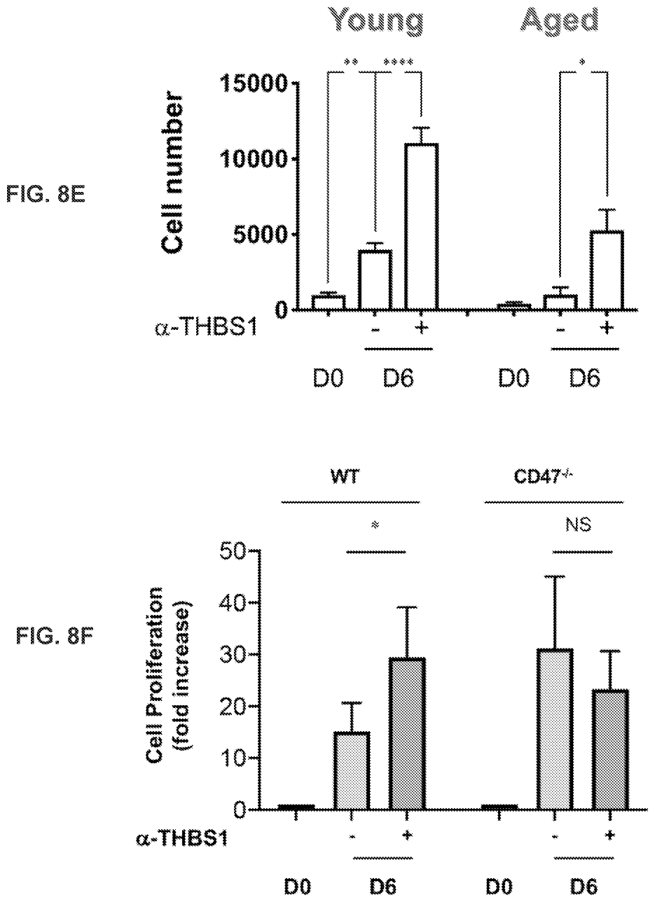

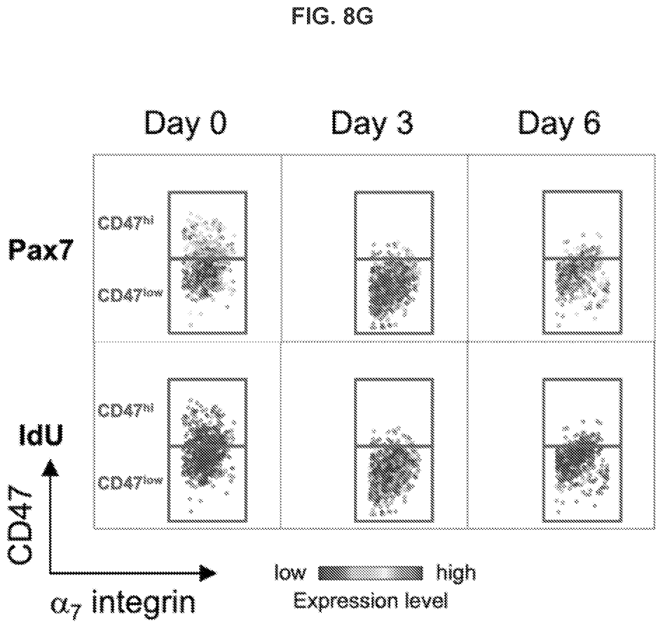

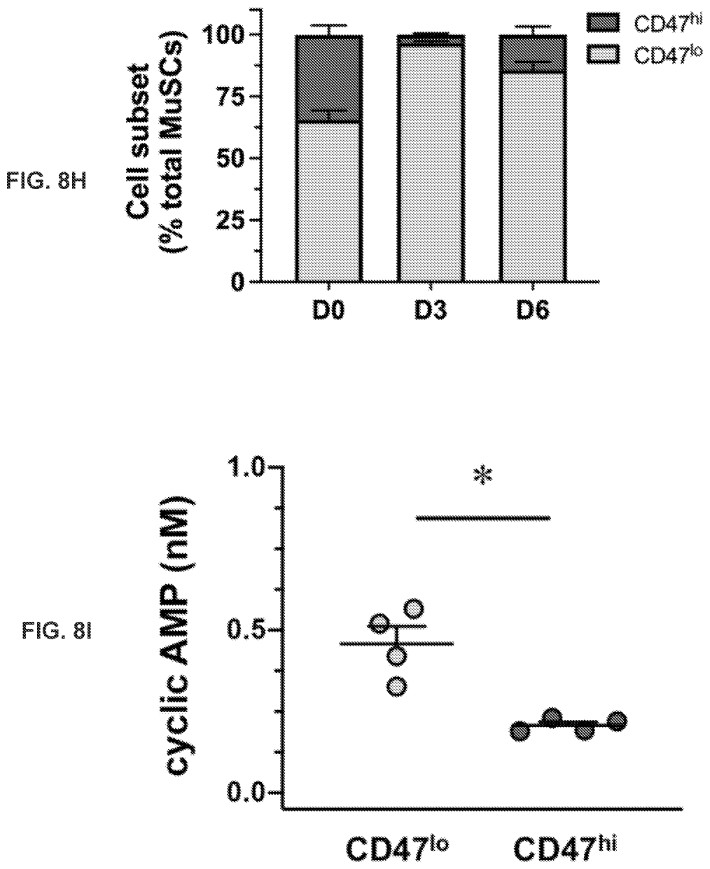

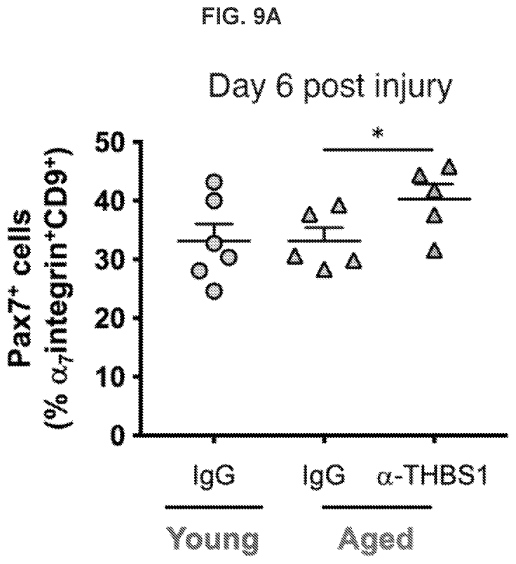

First, to determine whether exposure to thrombospondin-1 suppresses MuSC proliferation in vitro, cells were cultured for one week in the presence of increasing concentrations of thrombospondin-1. We found that as the dose of thrombospondin-1 increased MuSC numbers decreased, demonstrating that thrombospondin-1 suppresses MuSC proliferation in vitro in a CD47 dependent manner (FIG. 3B). An in vitro iododeoxyuridine (IdU) incorporation assay followed by multiparametric CyTOF analysis confirmed that the decrease in cell number was due to a decrease in cell proliferation, as shown by a decrease in the fraction of both IdU+ cells as well and ki-67+ cells (FIGS. 3C, 8A). To determine whether thrombospondin-1 treatment caused apoptosis we stained the cultured cells with Cleaved PARP. Low abundance of Cleaved PARP+ cells in both untreated and thrombospondin-1 treated MuSCs ruled out that thrombospondin-1 treatment led to an apoptosis (FIG. 8B). Given the role of thrombospondin-1 in suppression of cell proliferation we set out to investigate the downstream targets of thrombospondin-1 signaling. Based on previous findings we focused on cell cycle inhibitors that belong to the family of CDK interacting protein/Kinase inhibitor protein (Cip/Kip CKI). We performed q-RT-PCR for p21 (CDKN1a), p27 (CDKN1b), p57 (CDKN1c) and found that the mRNA expression of all three increased significantly 21 hours after thrombospondin-1 treatment in vitro (FIG. 8D). CyTOF analysis of p57, six days post thrombospondin-1 treatment showed that p57 protein levels also increased (FIG. 8C).

To assess whether thrombospondin-1 blockade could mitigate the proliferative defect of aged MuSCs in vitro, we treated them with a thrombospondin-1 antibody that blocks its interaction with CD47¹² and found that MuSC proliferation was restored to the levels of untreated young MuSCs (FIG. 8E). The antibody used was A6.1 (Annis et al. J Thromb Haemost. 2006 February; 4(2): 459-46, and its specificity was confirmed using CD47 knockout cells and mice: the effects of the THBS1 antibody (increased proliferation, IdU incorporation) were abrogated in CD47 knock out cells (FIGS. 3D, 8F). To establish the target of thrombospondin-1, we sorted CD47^{lo} and CD47^{hi} MuSC subsets from young and aged mice and treated them in vitro with the blocking antibody to thrombospondin-1 for one week. Strikingly, the CD47^{lo} MuSC subset isolated from young and aged mice exhibited increased proliferation upon treatment with the blocking antibody, while the CD47^{hi} subset did not expand (FIGS. 3E, 3F).

These findings demonstrate that thrombospondin-1 suppresses the proliferation of CD47^{lo} MuSCs in vitro, suggesting that MuSC subsets that express different levels of CD47 exhibit different sensitivities to thrombospondin-1 mediated inhibition of proliferation. We hypothesized that these MuSC subsets differ in their cell cycle status or in their signaling response to thrombospondin-1. This could lead to activation of different signaling pathways in response to thrombospondin-1 stimulation and determination of cell fates (self-renewal or commitment).

To determine whether the two MuSC subsets differed m their cell cycle status or their commitment to the myogenic program we performed phenotypic single-cell analysis by CyTOF during an in vivo time course of recovery from acute injury. We found that the CD47^{lo} MuSC subset that expressed high levels of Pax7 prior to injury proliferated upon injury, as measured by increased incorporation of iododeoxyuridine (IdU) (FIG. 8G). By contrast, the CD47^{hi} MuSC subset that expressed low levels of Pax7 did not expand upon injury (FIGS. 8G, 8H). These findings suggest that CD47^{lo} and CD47^{hi} MuSCs represent self-renewing stem cells and their committed progeny, respectively.

To address whether MuSC subsets exhibited a differential signaling response to thrombospondin-1 stimulation, we focused on cAMP signaling, a key regulator of cell proliferation^{44,45}. Thrombospondin-1 has previously been shown to decrease cAMP levels through activation of Gᵢ proteins^{46,47}. To determine whether CD47^{lo} and CD47^{hi} MuSC subsets differed in their ability to modulate cAMP signaling in response to thrombospondin-1 stimulation, we employed live single-cell confocal imaging of sorted MuSC subsets expressing a fluorescent cAMP downward sensor⁴⁸ (FIG. 3E). Using this assay, an increased GFP signal is a readout for decreased cAMP levels (FIG. 3E). CD47^{lo} and CD47^{hi} MuSCs were isolated from young mice and transfected with a baculovirus encoding the cAMP downward sensor. MuSC subsets were then treated with thrombospondin-1 and individually imaged every 10 sec for 5 minutes. Upon treatment of the CD47^{lo} MuSC subset with thrombospondin-1, the GFP signal increased. This was not the case for the CD47^{hi} MuSC subset under the same conditions (FIG. 3F), demonstrating that in CD47^{hi} MuSCs thrombospondin-1 does not signal through cAMP. We measured the basal cAMP levels in sorted CD47^{lo} and CD47^{hi} subsets and found that CD47^{lo} MuSC subsets exhibited significantly higher levels of cAMP compared to CD47^{hi} (FIG. 8I) In summary, the phenotypic analysis of the MuSC subsets, together with the cAMP signaling studies indicate that the CD47^{hi} and CD47^{lo} MuSCs subsets are both functionally and molecularly distinct.

To establish the source of thrombospondin-1 in vivo in skeletal muscle, we capitalized on single-cell mass cytometry and simultaneously stained young and aged muscle cells on the surface with antibodies against markers that distinguish distinct cell types and intracellularly with an antibody against thrombospondin-1. We found that the CD47^{hi} MuSC subset expressed higher levels of thrombospondin-1 than the CD47^{lo} (FIG. 3G). To investigate whether thrombospondin-1 had a physiological role during regeneration, we extended our analysis of thrombospondin-1 expression to cells within the Lin⁻/α₇integrin⁺/CD9⁺ myogenic compartment, which includes, in addition to the stem cells (Lin⁻/α₇integrin/CD9^{int}), distinct progenitor populations (Lin⁻/α₇integrin/CD9^{hi}) P1 and P2, distinguished by co-expression of CD9 and CD104 as we described previously²⁶. Strikingly, we found that P1 progenitor cells expressed significantly higher levels of both thrombospondin-1 and CD47 compared to MuSCs (FIG. 3H).

We hypothesized that during regeneration, after MuSCs expand, their P1 progeny secretes thrombospondin-1 in a negative feedback loop to prevent stem cell exhaustion and promote MuSC return to quiescence. To test this, we investigated the dynamics of thrombospondin-1 expression in vivo, in the context of recovery from acute injury. We performed an injury time course by notexin injection and measured thrombospondin-1 expression intracellularly in muscle stem and progenitor cells by CyTOF. Thrombospondin-1 expression was low in MuSCs from young mice compared to P1 progenitor cells and did not significantly change over the injury time course (FIG. 3I). However, thrombospondin-1 expression in young P1 progenitor cells changed dynamically throughout the injury time course. It decreased significantly at day 3 post injury, a time during which MuSCs expand dramatically²⁶, and returned to a level similar to the resting state at day 6 post injury (FIG. 3I). By contrast, thrombospondin-1 expression in the aged myogenic compartment exhibited a marked dysregulation. In aged MuSCs thrombospondin-1 expression levels increased at day 3 post injury, and decreased by day 6 (FIG. 3J). In aged progenitor cells, thrombospondin-1 expression levels did not change (FIG. 3J). The dynamic change in thrombospondin-1 expression in young progenitor cells supports a “quorum sensing model”⁴⁹ in which the progenitor cell population density influences MuSC expansion. We propose that the population density of progenitor cells can be sensed by MuSCs through the secreted protein thrombospondin-1 that, when released by progenitor cells after MuSC expansion, acts in a paracrine fashion to suppress proliferation of the neighboring MuSCs and promote their return to quiescence.

### Thrombospondin-1 Blockade In Vivo Activates Muscle Stem Cells in the Absence of Injury and Enhances the Regenerative Response of Aged Muscle.

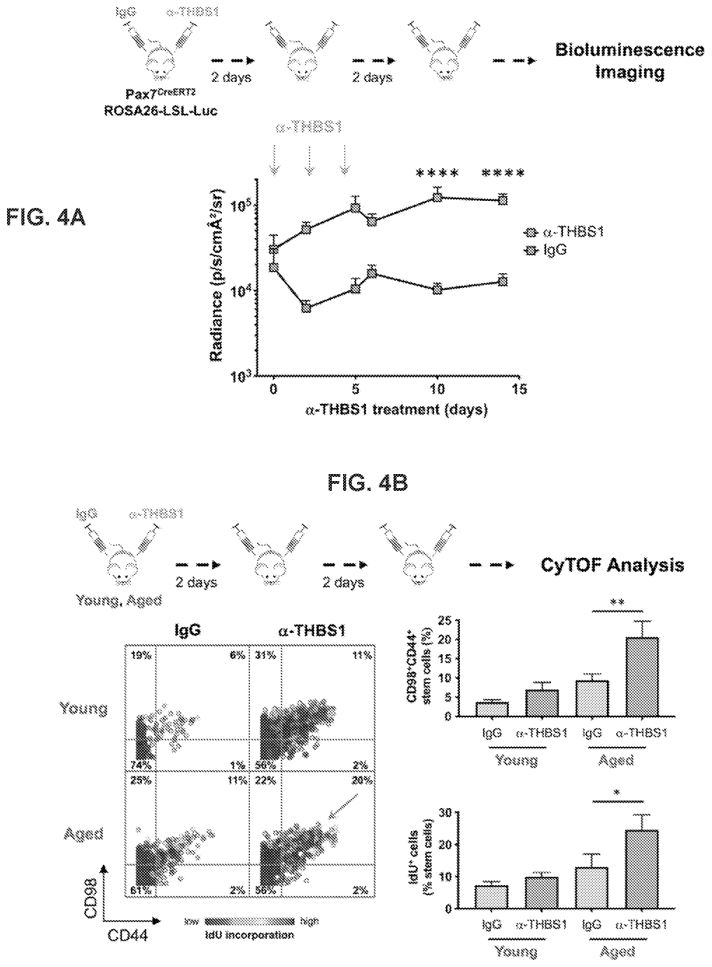

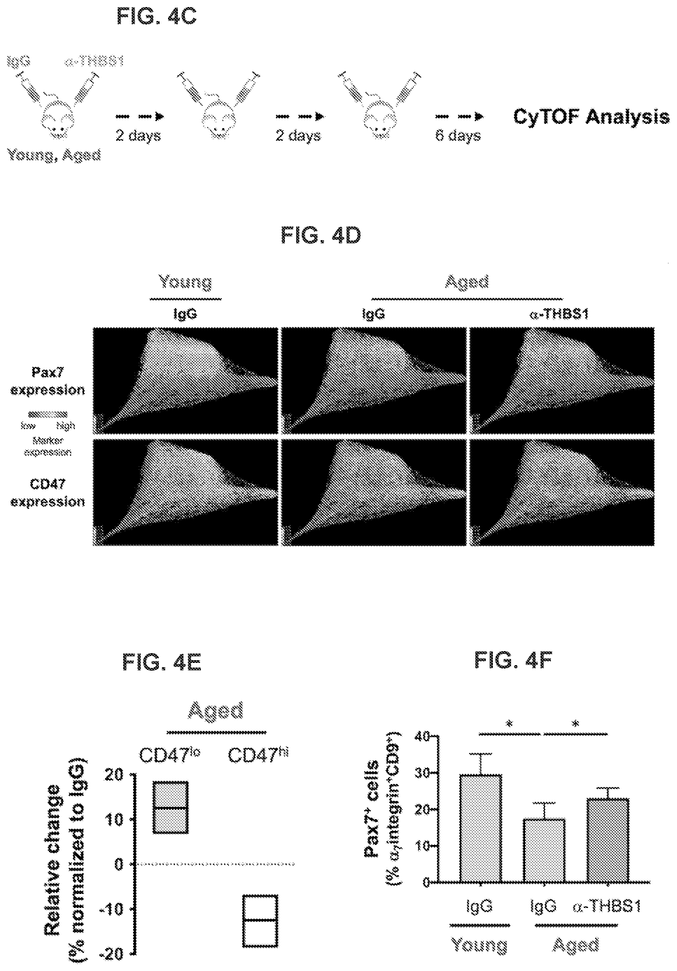

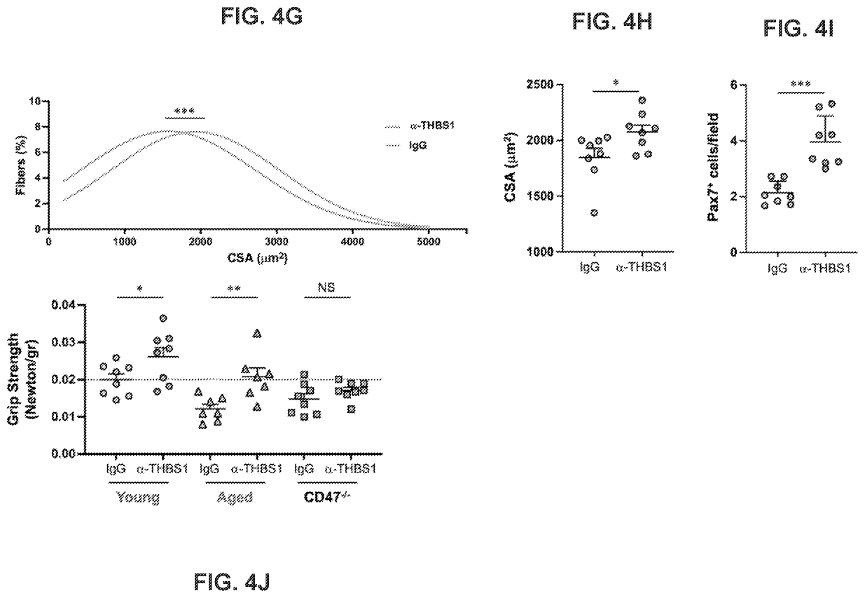

To test the quorum sensing model in our system in vivo, we treated Pax7^{CreERT2}; Rosa26-LSL-Luc mice, a transgenic mouse model in which endogenous MuSC numbers can monitored by bioluminescence imaging, with a thrombospondin-1 blocking antibody or IgG control regimen, which consisted of three intramuscular (i.m.) injections in the TA muscle at two-day intervals (FIG. 4A, scheme). Remarkably, in vivo treatment with the thrombospondin-1 blocking antibody was sufficient to significantly expand MuSCs in the absence of injury compared to the IgG control (FIG. 4A), suggesting that thrombospondin-1 plays a role in preventing MuSC activation during homeostasis.

To investigate the magnitude of MuSC activation and proliferation in young and aged mice upon thrombospondin-1 blockade in vivo, we treated mice with the same antibody regimen used above in the TA and GA muscles and employed multiparametric CyTOF analysis of skeletal muscle to measure IdU incorporation in the previously defined activated MuSC subset²⁶ (CD44⁺/CD98⁺, FIG. 4B). Strikingly, aged mice that received the thrombospondin-1 blockade exhibited a two-fold increase in the proportion of activated stem cells (FIG. 4B, biaxial plots). Moreover, they showed a significant increase in the proportion of stem cells that incorporated IdU, shown by color overlay (FIG. 4B, biaxial plots), suggesting that in vivo thrombospondin-1 blockade boosts the proliferative capacity of aged MuSCs in resting muscle.

To determine whether activated aged MuSCs returned to quiescence following in vivo thrombospondin-1 blockade, hindlimb muscles from aged mice treated with the anti-thrombospondin-1 regimen were collected 6 days after the end of treatment (FIG. 4C). High-resolution CyTOF analysis of the entire myogenic compartment, using X-shift clustering paired with single-cell force directed layout visualization, revealed that aged mice treated with control IgG exhibited a significant decrease in the proportion of Pax7^{hi} stem cells in their TA and GA muscles compared to young mice (FIGS. 4D, 4F). Strikingly, thrombospondin-1 blockade was sufficient to increase the number of Pax7^{hi} stem cells to a level similar to that seen in young mice (FIGS. 4D, 4F). Moreover, the Pax7^{hi} stem cells in treated aged mice expressed lower levels of CD47 compared to control, suggesting that thrombospondin-1 blocking antibody facilitated the expansion of CD47^{lo} MuSCs in aged mice (FIGS. 4D, 4E). We complemented our in vivo antibody treatment studies with physiological analyses and performed muscle strength tests and detailed histological analyses. The treatment resulted in an increase in muscle cross-sectional area and in the number of Pax7+ cells compared to IgG treated samples (FIGS. 4G-I). Moreover thrombospondin-1 blockade led to an increase in grip strength in both young and aged animals compared to the IgG treated animals (FIG. 4J).

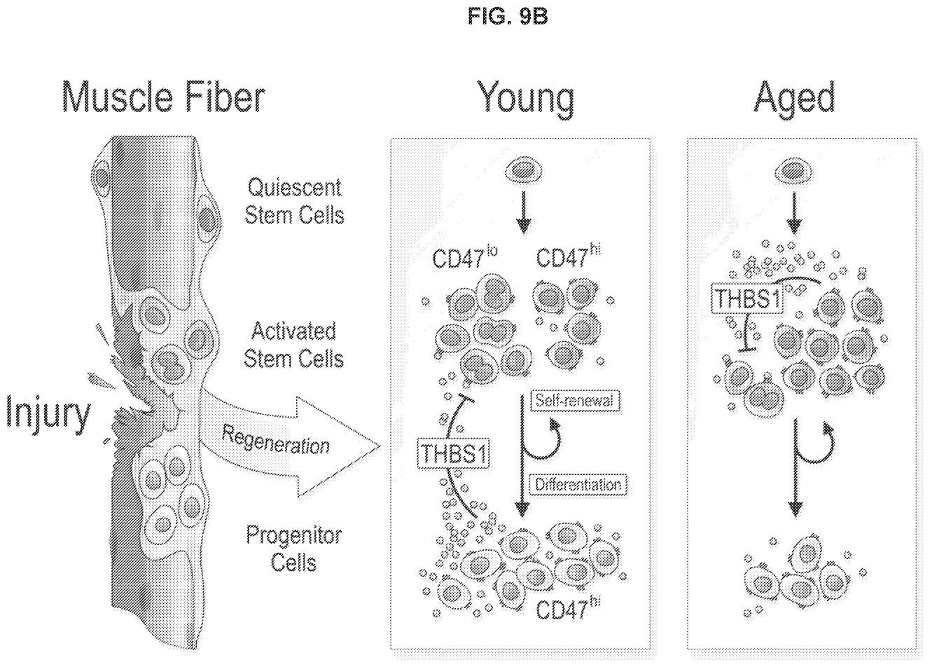

To determine whether thrombospondin-1 blockade could enhance the regenerative response of aged MuSCs in vivo, TA and GA muscles of young and aged mice were treated with the anti-thrombospondin-1 regimen described above and then acutely injured by notexin injection. Hindlimb muscles were harvested 3- and 6-days post injury and analyzed by single-cell mass cytometry to quantify MuSC expansion at day 3 and Pax7 expression at day 6. We found that at day 3 post injury, when MuSC expansion is normally at its peak^{7,26}, control IgG-treated aged MuSCs, defined as Lin⁻/α₇integrin⁺/CD9^{int}, comprised a smaller fraction of the myogenic compartment than young MuSCs (FIGS. 5A-5B). Strikingly, in vivo thrombospondin-1 blockade led to a significant increase in the proportion of aged MuSCs at day 3 post injury, compared to control (FIG. 5B), as well as an increase in the proportion of aged activated (CD98⁺/CD44⁺) and proliferating (IdU⁺) MuSCs (FIG. 5C), suggesting improved regenerative capacity. Moreover, thrombospondin-1 blockade was sufficient to increase the number of Pax7^{hi} cells in the aged myogenic compartment at day 6 post injury (FIG. 9A), suggesting that the expanded aged MuSC population was able to self-renew. To determine whether thrombospondin-1 blockade in vivo could accelerate recovery from injury we performed grip strength tests and force measurement analysis in vivo. We found that treated mice exhibited a significant increase in both grip strength and force at day 10 post injury (FIGS. 5D,E). No significant difference in these parameters was found in CD47−/− mice confirming the specificity of the blocking antibody. Our data indicate that modulation of thrombospondin-1 signaling in vitro and in vivo by a thrombospondin-1 antibody treatment represents a promising therapy to restore the regenerative potential of aged MuSCs.

c) Discussion

The loss of muscle mass and strength with aging is a major predictor of poor health outcome^{3,50} and sarcopenia is now recognized as a disease by the World Health Organization⁵¹. Muscle function is essential to the preservation of mobility and independence with age and its loss is associated with billions of healthcare dollars^{5,6}. Sarcopenia is due in part to the accumulation of dysfunctional stem cells that have lost the ability to proliferate, maintain and regenerate the tissue²⁰⁻²³. Previous studies from our group and others have identified cell intrinsic and extrinsic signaling pathways that are dysregulated in aged MuSCs, leading to defects in quiescence, self-renewal and proliferation¹⁵⁻²³. However, a major barrier to elucidating the molecular mechanisms responsible for the age-associated decline in MuSC regenerative capacity has been the heterogeneity of the MuSC population, underscoring the need for single-cell studies. To resolve the molecular and functional heterogeneity of the MuSC population and distinguish dysfunctional subsets that can be purified and characterized during aging, we capitalized on our previously described cell surface marker screen²⁶ and single-cell mass cytometry^{25,27}. Here we uncover a new role for the surface marker CD47 as a regulator of skeletal muscle cell function. CD47 is a transmembrane protein that belongs to the immunoglobulin superfamily^{52,53} and has been extensively studied in the context of hematopoiesis, where it functions as both a receptor and a ligand. It is a ligand for SIRPα through which CD47 prevents phagocytosis⁴³ and a receptor for the extracellular matrix protein thrombospondin-1^{54,55}. The function of CD47 in stem cell fate in other tissues has been understudied. Our studies highlight a new role for CD47 levels in determining muscle stem cell fate: self-renewal or commitment.

While CD47 is expressed on all cells, including erythrocytes^{56,57}, its expression levels are transiently regulated in different contexts^{28,58,59}, modulating its function. Previous studies have shown that CD47 expression is increased on the surface of hematopoietic stem cells (HSCs) upon damage-induced mobilization and that this increase protects HSCs in the periphery from being cleared by phagocytes during immune surveillance²⁸. Elevated expression of CD47 on CD4 T cells has been shown to define functional long-lived memory T cell precursors⁵⁸. Finally, studies in red blood cells, which exhibit a short lifespan, have shown that decreased CD47 levels mediate age-related clearance of these cells⁵⁹, providing evidence for regulation of CD47 expression with age. However, a role for CD47 in regeneration and aging has not previously been reported in muscle.

We found that expression of CD47 on the cell membrane distinguishes a dysfunctional CD47^{hi} MuSC subset that accumulates in aged muscle, from a CD47^{lo} MuSC subset with superior regenerative potential that is enriched in young muscle. Alternative polyadenylation of CD47 mRNA has been proposed as a regulatory mechanism for CD47 protein expression in human cell lines^{33,34}. Here we identify multiple CD47 mRNA isoforms with 3′UTRs of different lengths in muscle stem and progenitor cells. Alternative polyadenylation has previously been shown to play a role in modulating MuSC function in different muscles, by generating isoforms with different susceptibility to miRNA binding, leading to translational repression¹³. In our system, alternative polyadenylation site selection generates CD47 mRNA isoforms with different susceptibility to binding of a protein complex that leads to differential subcellular localization³³. Specifically, we found that in young muscle expression of the CD47 mRNA isoform with the long 3′UTR is elevated at the stem to progenitor cell transition, leading to a switch from an intracellular to a membrane spanning form of CD47. By contrast, in aged muscle the CD47 mRNA isoform with the long 3′UTR is increased prematurely in MuSCs, leading to the accumulation of the CD47^{hi} committed MuSC subset. These findings underscore the importance of post-transcriptional control in the regulation of stem cell fate.

To understand the role of CD47 in the regulation of MuSC fate and function, we focused on investigating its interaction with thrombospondin-1, as this pathway has previously been implicated in self-renewal and reprogramming^{30,60}. In young muscle in the context of injury we found that progenitor cells secrete thrombospondin-1 during the late stages of regeneration, establishing a negative feedback loop that limits MuSC expansion and promotes MuSC return to quiescence. In aged muscle we discovered that the dysfunctional CD47^{hi} MuSC subset precociously secretes thrombospondin-1 in the early stages of regeneration to inhibit the expansion of the CD47^{lo} MuSC subset, thus impairing aged muscle regeneration. Importantly, we showed that transient thrombospondin-1 blockade in two different in vivo contexts, the resting state and acute muscle injury, was sufficient to promote the activation and self-renewal of the aged CD47^{lo} MuSC subset and enhance aged muscle regenerative response.

In summary, the work presented here reveals a MuSC subset that accumulates during aging and uncovers a previously unrecognized signaling loop that underlies aged MuSC dysfunction. Taken together, our work sheds light on mechanisms that can be exploited to overcome sarcopenia and may underlie age-related stem cell dysfunction more generally. Our work provides fresh insights into the role of CD47 and suggests a new therapeutic strategy, immune blockade, to tackle the loss of regenerative function in aging.

### Example 2. Results in Dystrophic Mice and MuSCs

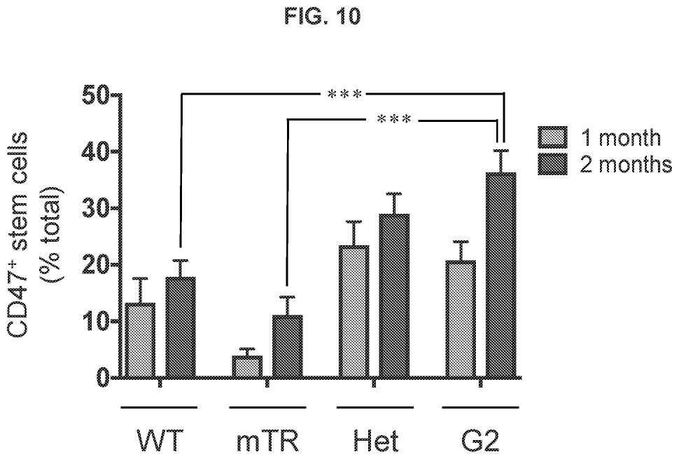

CD47 protein expression in dystrophic (Mdx-mTR G2 (G2)) and control (WT, mTR and Het) muscle stem cells was measured by CyTOF analysis at one and two months of age. The graph (FIG. 10) shows that the fraction of CD47+ stem cells significantly increases in dystrophic muscle stem cells.

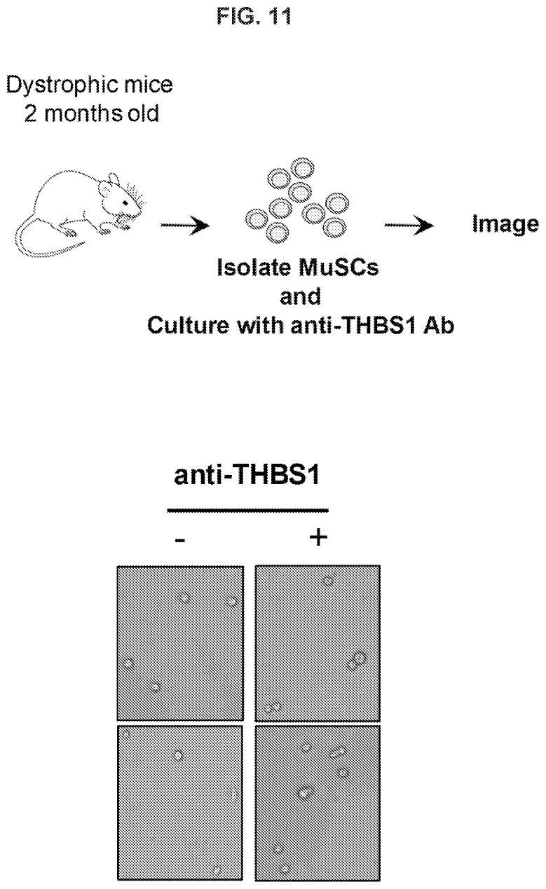

a₇integrin⁺/CD34⁺ MuSCs sorted from dystrophic G2 mice were cultured in growth media for 6 days on biomimetic hydrogels in the presence (+) or absence (−) of a blocking antibody to THBS1 and changes in proliferation was monitored by microscopy analysis. Thrombospondin-1 blockade led to increased proliferation of G2 MuSCs (FIG. 11).

### REFERENCES

1. von Haehling, S., Morley, J. E. & Anker, S. D. An overview of sarcopenia: Facts and numbers on prevalence and clinical impact. Journal of Cachexia, Sarcopenia and Muscle (2010). doi:10.1007/s13539-010-0014-2
2. Martinez, B. P. et al Frequency of sarcopenia and associated factors among hospitalized elderly patients. BMC Musculoskelet. Disord. (2015). doi:10.1186/s12891-015-0570-x
3. Rockwood, K. & Mitnitski, A. Frailty in relation to the accumulation of deficits. Journals of Gerontology—Series A Biological Sciences and Medical Sciences (2007). doi:10.1093/gerona/62.7.722
4. Robertson, D. A., Savva, G. M. & Kenny, R. A. Frailty and cognitive impairment-A review of the evidence and causal mechanisms. Ageing Research Reviews (2013). doi:10.1016/j.arr.2013.06.004
5. Rolland, Y. et al. Sarcopenia: Its assessment, etiology, pathogenesis, consequences and future perspectives. Journal of Nutrition, Health and Aging (2008). doi:10.1007/BF02982704
6. Goates. S. et al. Economic Impact of Hospitalizations in US Adults with Sarcopenia. J. frailty aging (2019). doi:10.14283/jfa.2019.10
7. Blau, H. M., Cosgrove, B. D. & Ho, A. T. V. The central role of muscle stem cells in regenerative failure with aging. Nat. Publ. Gr. 21, 854-862 (2015).
8. Sousa-Victor, P. & Muñoz-Cánoves, P. Regenerative decline of stem cells in sarcopenia. Molecular Aspects of Medicine (2016). doi:10.1016/j.mam.2016.02.002
9. Feige, P., Brun, C. E., Ritso, M. & Rudnicki, M. A. Orienting Muscle Stem Cells for Regeneration in Homeostasis, Aging, and Disease. Cell Stem Cell (2018). doi:10.1016/j.stem.2018.10.006
10. MAURO, A. Satellite cell of skeletal muscle fibers. J. Biophys. Biochem. Cytol. (1961). doi:10.1083/jcb.9.2.493
11. Keefe, A. C. et al. Muscle stem cells contribute to myofibres in sedentary adult mice. Nat. Commun. (2015). doi:10.1038/ncomms8087
12. Pawlikowski, B., Pulliam, C., Betta, N. D., Kardon, G. & Olwin, B. B. Pervasive satellite cell contribution to uninjured adult muscle fibers. Skelet. Muscle (2015). doi:10.1186/s133954015-0067-1
13. de Morree, A. et al. Alternative polyadenylation of Pax3 controls muscle stem cell fate and muscle function. Science (80-). 366, 734-738 (2019).
14. Schmidt, M., Schüler, S. C., Hüttner, S. S., von Eyss, B. & von Maltzahn, J. Adult stem cells at work: regenerating skeletal muscle. Cellular and Molecular Life Sciences (2019). doi:10.1007/s00018-019-03093-6
15. Lukjanenko, L. et al. Loss of fibronectin from the aged stem cell niche affects the regenerative capacity of skeletal muscle in mice. Nat. Med. (2016). doi:10.1038/nm.4126
16. Lukjanenko, L. et al. Aging Disrupts Muscle Stem Cell Function by Impairing Matricellular WISP1 Secretion from Fibro-Adipogenic Progenitors. Cell Stem Cell (2019). doi:10.1016/j.stem.2018.12.014
17. Chakkalakal, J. V., Jones, K. M., Basson, M. A. & Brack, A. S. The aged niche disrupts muscle stem cell quiescence. Nature (2012). doi:10.1038/naturel 1438
18. Vinel, C. et al. The exerkine apelin reverses age-associated sarcopenia. Nat. Med. (2018). doi:10.1038/s41591-018-0131-6
19. Rozo, M., Li, L. & Fan, C. M. Targeting β1-integrin signaling enhances regeneration in aged and dystrophic muscle in mice. Nat. Med. (2016). doi:10.1038/nm.4116
20. Cosgrove, B. D. et al. Rejuvenation of the muscle stem cell population restores strength to injured aged muscles. Nat. Med. 20, 255-264 (2014).
21. Bernet, J. D. et al. p38 MAPK signaling underlies a cell-autonomous loss of stem cell self-renewal in skeletal muscle of aged mice. Nat. Med. 20, (2014).
22. Tierney. M. T. et al. STAT3 signaling controls satellite cell expansion and skeletal muscle repair. Nat. Med. (2014). doi:10.1038/nm.3656
23. Price, F. D. et al. Inhibition of JAK-STAT signaling stimulates adult satellite cell function. 20, (2014).
24. Brett, J. O. et al. Exercise rejuvenates quiescent skeletal muscle stem cells in old mice through restoration of Cyclin D1. Nat. Metab. 2, 307-317 (2020).
25. Spitzer, M. H. & Nolan, G. P. Mass Cytometry: Single Cells, Many Features. Cell 165, 780-791 (2016).
26. Porpiglia, E. et al. High-resolution myogenic lineage mapping by single-cell mass cytometry. Nat. Cell Biol. 19, 558-567 (2017).
27. Bjornson, Z. B., Nolan, G. P. & Fantl, W. J. Single-cell mass cytometry for analysis of immune system functional states. Current Opinion in Immunology (2013). doi:10.1016/j.coi.2013.07.004
28. Jaiswal, S. et al. CD47 Is Upregulated on Circulating Hematopoietic Stem Cells and Leukemia Cells to Avoid Phagocytosis. Cell (2009). doi:10.1016/j.cell.2009.05.046
29. Gao, Q., Chen, K., Gao, L., Zheng, Y. & Yang, Y. G. Thrombospondin-1 signaling through CD47 inhibits cell cycle progression and induces senescence in endothelial cells. Cell Death Dis. 7, e2368 (2016).
30. Kaur, S. et al. Thrombospondin-1 signaling through CD47 inhibits self-renewal by regulating c-Myc and other stem cell transcription factors. Sci. Rep. (2013). doi:10.1038/srep01673
31. Feng, N. et al. MiR-487b promotes human umbilical vein endothelial cell proliferation, migration, invasion and tube formation through regulating THBS1. Neurosci. Lett. (2015). doi:10.1016/j.neulet.2015.02.002
32. Annis, D. S., Murphy-Ullrich, J. E. & Mosher. D. F. Function-blocking antithrombospondin-1 monoclonal antibodies. J. Thromb. Haemost. (2006). doi:10.1111/j.1538-7836.2006.01723.x
33. Berkovits, B. D. & Mayr, C. Alternative 3′ UTRs act as scaffolds to regulate membrane protein localization. Nature (2015). doi:10.1038/nature14321
34. Ma, W. & Mayr, C. A Membraneless Organelle Associated with the Endoplasmic Reticulum Enables 3′UTR-Mediated Protein-Protein Interactions. Cell (2018). doi:10.1016/j.cell.2018.10.007
35. Apponi, L. H., Corbett, A. H. & Pavlath, G. K. RNA-binding proteins and gene regulation in myogenesis. Trends in Pharmacological Sciences (2011). doi:10.1016/j.tips.2011.06.004
36. Figueroa. A. et al. Role of HuR in Skeletal Myogenesis through Coordinate Regulation of Muscle Differentiation Genes. Mol. Cell. Biol. (2003). doi:10.1128/mcb.23.14.4991-5004.2003
37. van der Giessen, K. & Gallouzi, I.-E. Involvement of Transportin 2-mediated HuR Import in Muscle Cell Differentiation. Mol. Biol. Cell (2007). doi:10.1091/mbc.e07-02-0167
38. Hoque, M. et al. Analysis of alternative cleavage and polyadenylation by 3′ region extraction and deep sequencing. Nat. Methods (2013). doi:10.1038/nmeth.2288
39. van Buuren, N., Tellinghuisen, T. L., Richardson, C. D. & Kirkegaard, K.

Transmission genetics of drug-resistant hepatitis C virus. Elife (2018). doi:10.7554/eLife.32579
40. Lindberg, F. P. et al. Decreased resistance to bacterial infection and granulocyte defects in IAP-deficient mice. Science (80-). 274, 795-798 (1996).
41. Oldenborg, P.-A. CD47: A Cell Surface Glycoprotein Which Regulates Multiple Functions of Hematopoietic Cells in Health and Disease. ISRN Hematol. (2013). doi:10.1155/2013/614619
42. Majeti, R. et al. CD47 Is an Adverse Prognostic Factor and Therapeutic Antibody Target on Human Acute Myeloid Leukemia Stem Cells. Cell (2009). doi:10.1016/j.cell.2009.05.045
43. Brown, E. J. & Frazier. W. A. Integrin-associated protein (CD47) and its ligands. Trends Cell Biol. 11, 130-5 (2001).
44. Stork, P. J. S. & Schmitt, J. M. Crosstalk between cAMP and MAP kinase signaling in the regulation of cell proliferation. Trends in Cell Biology (2002). doi:10.1016/S0962-8924(02)02294-8
45. Dumaz, N. & Marais, R. Integrating signals between cAMP and the RAS/RAF/MEK/ERK signalling pathways: Based on the Anniversary Prize of the Gesellschaft far Biochemie und Molekularbiologie Lecture delivered on 5 Jul. 2003 at the Special FEBS Meeting in Brussels. in FEBS Journal (2005). doi:10.1111/j.1742-4658.2005.04763.x
46. Yao, M., Roberts, D. D. & Isenberg, J. S. Thrombospondin-1 inhibition of vascular smooth muscle cell responses occurs via modulation of both cAMP and cGMP. Pharmacol. Res. (2011). doi:10.1016/j.phrs.2010.10.014
47. Wang, X. Q., Lindberg, F. P. & Frazier, W. A. Integrin-associated protein stimulates α2β1-dependent chemotaxis via Gi-mediated inhibition of adenylate cyclase and extracellular-regulated kinases. J. Cell Biol. (1999). doi:10.1083/jcb.147.2.389
48. Tewson, P. H., Martinka. S., Shaner, N. C., Hughes. T. E. & Quinn, A. M. New DAG and cAMP Sensors Optimized for Live-Cell Assays in Automated Laboratories. J. Biomol. Screen. (2016). doi:10.1177/1087057115618608
49. Balagaddé, F. K., You, L., Hansen, C. L., Arnold, F. H. & Quake. S. R Microbiology: Long-term monitoring of bacteria undergoing programmed population control in a microchemostat. Science (80-). (2005). doi:10.1126/science.1109173
50. Landi, F. et al. Sarcopenia as a risk factor for falls in elderly individuals: Results from the ilSIRENTE study. Clin. Nutr. (2012). doi:10.1016/j.clnu.2012.02.007
51. Cao. L. & Morley. J. E. Sarcopenia Is Recognized as an Independent Condition by an International Classification of Disease, Tenth Revision, Clinical Modification (ICD-10-CM) Code. Journal of the American Medical Directors Association (2016). doi:10.1016/j.jamda.2016.06.001
52. Lindberg, F. P., Gresham, H. D., Schwarz, E. & Brown, E. J. Molecular cloning of integrin-associated protein: An immunoglobulin family member with multiple membrane-spanning domains implicated in α(v)β3-dependent ligand binding. J. Cell Biol. (1993). doi:10.1083/jcb.123.2.485
53. Lindberg. F. P. et al. Rh-related antigen CD47 is the signal-transducer integrin-associated protein. J. Biol. Chem. (1994).
54. Liu, Y. et al. The role of CD47 in neutrophil transmigration: Increased rate of migration correlates with increased cell surface expression of CD47. J. Biol. Chem. (2001). doi:10.1074/jbc.M104138200
55. Gao. A. G. et al. Integrin-associated protein is a receptor for the C-terminal domain of thrombospondin. J. Biol. Chem. (1996). doi:10.1074/jbc.271.1.21
56. Reinhold, M. I. et al. In vivo expression of alternatively spliced forms of integrin-associated protein (CD47). J. Cell Sci. (1995).
57. Oldenborg, P. A. et al. Role of CD47 as a marker of self on red blood cells. Science (80-). (2000). doi:10.1126/science.288.5473.2051
58. Van, V. Q. et al. CD47 high Expression on CD4 Effectors Identifies Functional Long-Lived Memory T Cell Progenitors. J. Immunol. 188, 4249-4255 (2012).
59. Khandelwal. S., Van Rooijen, N. & Saxena. R. K. Reduced expression of CD47 during murine red blood cell (RBC) senescence and its role in RBC clearance from the circulation. Transfusion (2007). doi:10.1111/j.1537-2995.2007.01348.x
60. Soto-Pantoja, D. R., Kaur, S. & Roberts, D. D. CD47 signaling pathways controlling cellular differentiation and responses to stress. Crit. Rev. Biochem. Mol. Biol. 50, 212-230 (2015).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for increasing muscle mass, strength, and/or regeneration in an aged, atrophied, or dystrophic skeletal muscle in a subject, the method comprising:
administering to the aged, atrophied, or dystrophic skeletal muscle a thrombospondin-1 inhibitor in an amount sufficient to inhibit binding of thrombospondin-1 to CD47 on the surface of one or more muscle stem cells (MuSCs) and/or reduce thrombospondin-1 levels in one or more MuSCs in the aged, atrophied, or dystrophic skeletal muscle, thereby increasing muscle mass, strength, and/or regeneration in the aged, atrophied, or dystrophic skeletal muscle.

2. The method of claim 1, wherein the subject has sarcopenia.

3. The method of claim 1 or 2, wherein the subject has one or more biomarkers of aging.

4. The method of claim 3, wherein the one or more biomarkers of aging is selected from the group consisting of: decreased muscle mass and/or strength relative to a level present in young skeletal muscle, decreased MuSC proliferation or activation relative to a level present in young skeletal muscle, increased CD47 surface expression in MuSCs relative to a level present in young skeletal muscle, and decreased levels of Pax7 in MuSCs relative to a level present in young skeletal muscle.

5. The method of claim 1, wherein the subject has a condition or disease associated with muscle atrophy.

6. The method of claim 5, wherein the condition or disease is spinal muscular atrophy, diabetes, frailty, sarcopenic obesity, neuropathy, or cachexia, or wherein the subject has muscle atrophy due to immobilization or muscle disuse.

7. The method of claim 1, wherein the subject has a muscular dystrophy.

8. The method of claim 7, wherein the muscular dystrophy is selected from the group consisting of Duchenne muscular dystrophy (DMD), Becker muscular dystrophy, congenital muscular dystrophy, distal muscular dystrophy, Emery-Dreifuss muscular dystrophy, facioscapulohumeral muscular dystrophy, limb girdle muscular dystrophy, myotonic muscular dystrophy (MDD), and oculopharyngeal muscular dystrophy.

9. The method of any one of claims 1 to 8, wherein the aged, atrophied, or dystrophic skeletal muscle is injured.

10. The method of claim 9, wherein the subject is preparing to undergo surgery, is undergoing surgery, or has undergone surgery.

11. The method of any one of claims 1 to 8, wherein the aged, atrophied, or dystrophic skeletal muscle is uninjured.

12. The method of any one of claims 1 to 11, wherein the method results in an increase in muscle mass and/or regeneration relative to the level in the aged, atrophied, or dystrophic skeletal muscle prior to the administering of the thrombospondin-1 inhibitor.

13. The method of any one of claims 1 to 12, wherein the method results in an increase in muscle mass and/or regeneration of at least 10% relative to the level in the aged, atrophied, or dystrophic skeletal muscle prior to the administering of the thrombospondin-1 inhibitor.

14. The method of any one of claims 1 to 13, wherein the method results in an increase in muscle mass, strength, and/or regeneration in the aged, atrophied, or dystrophic skeletal muscle to a level substantially similar to a level present in young and/or non-dystrophic skeletal muscle.

15. The method of any one of claims 1 to 14, wherein the administration results in an increase in the proliferation and/or activity of MuSCs in the aged skeletal muscle.

16. The method of any one of claims 1 to 15, wherein the administration results in an increase in the proliferation and/or activity of MuSCs in the aged, atrophied, or dystrophic skeletal muscle of at least 10% relative to the level in the aged, atrophied, or dystrophic skeletal muscle prior to the administering of the thrombospondin-1 inhibitor.

17. The method of any one of claims 1 to 16, wherein the administration results in an increase in the proliferation and/or activity of MuSCs in the aged, atrophied, or dystrophic skeletal muscle to a level substantially similar to a level present in young and/or non-dytrophic skeletal muscle.

18. The method of any one of claims 1 to 17, wherein the administration results in a decrease in CD47 surface levels and/or an increase in Pax7 expression in MuSCs in the aged, atrophied, or dystrophic skeletal muscle.

19. The method of any one of claims 1 to 18, wherein the administration results in a decrease in CD47 surface levels and/or an increase in Pax7 expression in MuSCs in the aged, atrophied, or dystrophic skeletal muscle relative to the level in the aged, atrophied, or dystrophic skeletal muscle prior to the administering of the thrombospondin-1 inhibitor.

20. The method of any one of claims 1 to 19, wherein the administration results in a decrease in CD47 surface levels and/or an increase in Pax7 expression in MuSCs in the aged skeletal muscle of at least 10% relative to the level in the aged, atrophied, or dystrophic skeletal muscle prior to the administering of the thrombospondin-1 inhibitor.

21. The method of any one of claims 1 to 20, wherein the administration results in a decrease in CD47 surface levels in MuSCs in the aged, atrophied, or dystrophic skeletal muscle to a level substantially similar to a level present in young and/or non-dystrophic skeletal muscle.

22. The method of any one of claims 1 to 21, wherein the subject is a human.

23. The method of claim 22, wherein the human is over 30, 40, 50, 60, 70, or 80 years of age.

24. The method of claim 23, further comprising a step wherein the human is selected for treatment with the inhibitor of thrombospondin-1 based on his or her age.

25. The method of any one of claims 1 to 21, wherein the subject is a non-human mammal.

26. The method of claim 25, wherein the non-human mammal is a farm animal.

27. The method of any one of claims 1 to 24, wherein the inhibitor is a small molecule compound, a peptide, or a blocking antibody or antibody fragment.

28. The method of claim 27, wherein the blocking antibody or antibody fragment is a monoclonal antibody or fragment thereof.

29. The method of claim 27 or 28, wherein the antibody fragment is selected from the group consisting of Fab, F(ab′)₂, ScFv, diabody, and nanobody.

30. The method of any one of claims 1 to 29, wherein the inhibitor is an antisense oligonucleotide, microRNA, siRNA, shRNA, CRISPR gRNA, or messenger RNA.

31. The method of any one of claims 1 to 30, wherein administering the inhibitor of thrombospondin-1 comprises systemic administration.

32. The method of any one of claims 1 to 30, wherein administering the inhibitor of thrombospondin-1 comprises local administration.

33. The method of claim 32, wherein the local administration comprises intramuscular injection.

34. A method for regenerating a population of muscle cells in a subject having a condition or disease associated with muscle damage, injury, or atrophy, the method comprising:
administering to the subject a therapeutically effective amount of an inhibitor of thrombospondin-1, to increase the population of muscle cells and/or to enhance muscle function in the subject.

35. A method for treating a condition or disease associated with muscle damage, injury or atrophy in a subject in need thereof, the method comprising:
administering to the subject (i) a therapeutically effective amount of a thrombospondin-1 inhibitor, and a pharmaceutically acceptable carrier, and (ii) a population of isolated muscle cells, to treat the condition or disease associated with muscle damage, injury, or atrophy.

36. A method of treating muscle damage, muscle injury or muscle atrophy comprising: administering a therapeutically effective amount of a thrombospondin-1 inhibitor, to a subject in need by intramuscular administration.

37. A method of treating muscle damage, muscle injury or muscle atrophy comprising administering a therapeutically effective amount of a composition comprising a thrombospondin-1 inhibitor to a subject in need thereof, thereby treating said muscle damage, muscle injury or muscle atrophy.

38. A method for stimulating the proliferation of a population of isolated muscle cells, the method comprising:
culturing the population of isolated muscle cells with a thrombospondin-1 inhibitor.
